SEQUENCE LISTING (32 sequences)

SEQ ID NO: 1 - DNA, 23 bp, Artificial Sequence
  Trans OCT4 forward primer
gagaaggatgtggtccgagtgtg

SEQ ID NO: 2 - DNA, 24 bp, Artificial Sequence
  Trans OCT4 reverse primer
ccctttttctggagactaaataaa

SEQ ID NO: 3 - DNA, 26 bp, Artificial Sequence
  Trans SOX2 forwarde primer
ggcacccctggcatggctcttggctc

SEQ ID NO: 4 - DNA, 24 bp, Artificial Sequence
  Trans SOX2 reverse primer
ttatcgtcgaccactgtgctgctg

SEQ ID NO: 5 - DNA, 25 bp, Artificial Sequence
  Trans KLF4 forward primer
acgatcgtggccccggaaaaggacc

SEQ ID NO: 6 - DNA, 24 bp, Artificial Sequence
  Trans KLF4 reverse primer
ttatcgtcgaccactgtgctgctg

SEQ ID NO: 7 - DNA, 27 bp, Artificial Sequence
  Trans c-Myc forward primer
caacaaccgaaaatgcaccagccccag

SEQ ID NO: 8 - DNA, 24 bp, Artificial Sequence
  Trans c-Myc reverse primer
ttatcgtcgaccactgtgctgctg

SEQ ID NO: 9 - DNA, 25 bp, Artificial Sequence
  biOCT4-3 forward primer
atttgttttttgggtagttaaaggt

SEQ ID NO: 10 - DNA, 27 bp, Artificial Sequence
  biOCT4-3 reverse primer
ccaactatcttcatcttaataacatcc

SEQ ID NO: 11 - DNA, 27 bp, Artificial Sequence
  biOCT4-4 forward primer
ggatgttattaagatgaagatagttgg

SEQ ID NO: 12 - DNA, 25 bp, Artificial Sequence
  biOCT4-4 reverse primer
cctaaactccccttcaaaatctatt

SEQ ID NO: 13 - DNA, 25 bp, Artificial Sequence
  biNanog forward primer
tggttaggttggttttaaatttttg

SEQ ID NO: 14 - DNA, 26 bp, Artificial Sequence
  biNanog reverse primer
aacccacccttataaattctcaatta

SEQ ID NO: 15 - DNA, 20 bp, Artificial Sequence
  IGF2 forward primer
cagacccccaaattatcgtg

SEQ ID NO: 16 - DNA, 20 bp, Artificial Sequence
  IGF2 reverse primer
gccaagaaggtgagaagcac

SEQ ID NO: 17 - DNA, 24 bp, Artificial Sequence
  MSX1 forward primer
cgagaggaccccgtggatgcagag

SEQ ID NO: 18 - DNA, 24 bp, Artificial Sequence
  MSX1 reverse primer
ggcggccatcttcagcttctccag

SEQ ID NO: 19 - DNA, 26 bp, Artificial Sequence
  Brachyury forward primer
gccctctccctcccctccacgcacag

SEQ ID NO: 20 - DNA, 26 bp, Artificial Sequence
  Brachyury reverse primer
cggcgccgttgctcacagaccacagg

SEQ ID NO: 21 - DNA, 19 bp, Artificial Sequence
  Runx2 forward primer
cggcaaaatgagcgacgtg

SEQ ID NO: 22 - DNA, 19 bp, Artificial Sequence
  Runx2 reverse primer
caccgagcacaggaagttg

SEQ ID NO: 23 - DNA, 24 bp, Artificial Sequence
  CD31 forward primer
atcatttctagcgcatggcctggt

SEQ ID NO: 24 - DNA, 24 bp, Artificial Sequence
  CD31 reverse primer
atttgtggagggcgaggtcataga

SEQ ID NO: 25 - DNA, 24 bp, Artificial Sequence
  CD34 forward primer
aaatcctcttcctctgaggctgga

SEQ ID NO: 26 - DNA, 24 bp, Artificial Sequence
  CD34 reverse primer
aagaggcagctggtgataagggtt

SEQ ID NO: 27 - DNA, 24 bp, Artificial Sequence
  VE-cad forward primer
tggagaagtggcatcagtcaacag

SEQ ID NO: 28 - DNA, 23 bp, Artificial Sequence
  VE-cad reverse primer
tctacaatcccttgcagtgtgag

SEQ ID NO: 29 - DNA, 19 bp, Artificial Sequence
  KDR forward primer
acacctaaaaacccagcac

SEQ ID NO: 30 - DNA, 20 bp, Artificial Sequence
  KDR reverse primer
tctgattccttcttcttcat

SEQ ID NO: 31 - DNA, 19 bp, Artificial Sequence
  GAPDH forward primer
gaaggtgaaggtcggagtc

SEQ ID NO: 32 - DNA, 20 bp, Artificial Sequence
  GAPDH reverse primer
gaagatggtgatgggatttc

(19) United States
(12) Patent Application Publication
(10) Pub. No.: US 2015/0299665 A1
(43) Pub. Date: Oct. 22, 2015

(54) METHOD FOR REPROGRAMMING OF HUMAN DENTAL PULP CELL USING OCT4 AND SOX2 AND USE THEREOF

(71) Applicant: KOREA RESEARCH INSTITUTE OF BIOSCIENCE AND BIOTECHNOLOGY, Daejeon (KR)

(72) Inventors: Yee Sook Cho, Daejeon (KR); Chae Wha Yoo, Daejeon (KR); Hee Jun Na, Daejeon (KR)

(73) Assignee: KOREA RESEARCH INSTITUTE OF BIOSCIENCE AND BIOTECHNOLOGY, Daejeon (KR)

(21) Appl. No.: 14/340,991

(22) Filed: Jul. 25, 2014

(30) Foreign Application Priority Data
Apr. 21, 2014 (KR) 10-2014-0047671

(51) Int. Cl. C12N 5/074; A61K 35/12; C12N 5/071

(52) U.S. Cl. CPC C12N 5/0696; C12N 5/0692; C12N 5/069; C12N 5/0691; A61K 35/12; C12N 2501/603; C12N 2501/602; C12N 2500/30; C12N 2506/45; C12N 2501/155; C12N 2501/165; C12N 2501/998; C12N 2501/115; C12N 2506/00; C12N 2501/135

(57) ABSTRACT

The present invention relates to a method for producing endothelial progenitor cells, comprising a method for producing induced pluripotent stem cells by using only Oct4 and Sox2 as a reprogramming factor of human dental pulp cells, and differentiating endothelial progenitor cells from the induced pluripotent stem cells produced by the method for producing induced pluripotent stem cells. Moreover, the present invention relates to a method for producing endothelial cells, comprising differentiating endothelial cells from endothelial progenitor cells produced by the method for producing the endothelial progenitor cells. Additionally, the present invention relates to a method for producing smooth muscle cells, comprising differentiating smooth muscle cells from endothelial progenitor cells produced by the method for producing the endothelial progenitor cells.

Also, the present invention make a significant contribution to treatment of ischemia disease and heart disease because a patient's dental pulp cell-derived differentiated cells can be used for cell treatment as a result of the cell treatment effect of the endothelial progenitor cells in a mouse model induced to have hind limb ischemia or myocardial infarction.

### TECHNICAL FIELD

The present invention is related to a method for producing induced pluripotent stem cells from human dental pulp cells comprising using only two reprogramming factors, Oct4 and Sox2, and a method for producing endothelial progenitor cells, comprising differentiating induced pluripotent stem cells into endothelial progenitor cells, wherein the induced pluripotent stem cells are produced by the method for producing induced pluripotent stem cells. Moreover, the present invention relates to a method for producing endothelial cells, comprising differentiating endothelial progenitor cells into endothelial cells, wherein the endothelial progenitor cells are produced by the method for producing the endothelial progenitor cells. Additionally, the present invention relates to a method for producing smooth muscle cells, comprising differentiating endothelial progenitor cells into smooth muscle cells, wherein the endothelial progenitor cells are produced by the method for producing the endothelial progenitor cells.

The present invention also relates to the induced pluripotent stem cells having an enhanced rate of differentiation into endothelial progenitor cells, wherein the induced pluripotent stem cells are produced by the method for producing induced pluripotent stem cells, and to the endothelial progenitor cells having an enhanced rate of differentiation into smooth muscle cells or endothelial cells, wherein the endothelial progenitor cells are produced by the method for producing endothelial progenitor cells.

Additionally, the present invention relates to endothelial cells produced by the method for producing the endothelial cells, or smooth muscle cells produced by the method for producing the smooth muscle cells.

The present invention also relates to a composition for treating ischemic disease and/or heart disease, which includes the induced pluripotent stem cells, endothelial progenitor cells, endothelial cells, smooth muscle cells, or a combination thereof. Also, the present invention relates to a method for producing the composition for treating ischemic disease and/or heart disease and a method for treatment of ischemic disease and/or heart disease comprising administering the composition produced by the method to an individual requiring the composition.

### BACKGROUND ART

The tremendous potential of endothelial progenitor cells (EPCs) isolated from human adult tissues for therapeutic use has been demonstrated for the treatment of a wide range of diseases. Despite progress in adult stem cell technology, restricted access to cells, a limited number of functional cells, and cell heterogeneity are obstacles to the use of endothelial progenitor cells in the regenerative medicine and screening fields.

As a promising alternative, induced pluripotent stem cell (iPSC) technology (Cell, 126, 663-676, 2006; Cell, 131, 861-872, 2007), which enables the reprogramming of a wider variety of cell types isolated from the human body into embryonic stem cell-like pluripotent cells, offers a novel strategy for the patient-specific derivation of clinically applicable lineage-specific cells, such as EPCs. The effects of transplanted human iPSC-derived EPCs (hiPSC-EPCs) was examined in mouse models with hind limb ischemia and myocardial infarction. The therapeutic effects of hiPSC-EPCs were known to be related to secretion of growth factors comprising blood-derived cytokines, angiopoietin-1 secreted from implanted EPCs, vascular endothelial growth factor (VEGF)-A and C, and platelet-derived growth factor (PDGF)-AA (Arteriosclerosis, Thrombosis, and Vascular Biology, 31, 72-79, 2011). Although progress has been made in hiPSC technology, there is still the limitation of the use of hiPSC lines, which are used in fabrication of EPCs, needs to be overcome and additional research of reprogramming and differentiation is needed to obtain a high homogeneity, enforcement of potentials, functionality, high transplantation survival rate in vivo, and safety before clinical trials.

Meanwhile, a broad range of adult cell types has been applied for hiPSC generation, and the characteristics of cells used for manufacturing hiPSCs have a great effect on the whole reprogramming process, particularly selection of reprogramming factors, kinetics and efficiency of reprogramming, and more importantly, differentiation propensity of reprogrammed hiPSCs. Studies have demonstrated that somatic cell memory predisposes reprogrammed hiPSCs to preferentially differentiate into parental cell types versus other cell types are unrelated to parental cells (Nature, 467, 285-290, 2010; Cell Stem Cell, 9, 17-23, 2011). However, the best starting cells for inducing endothelial progenitor cells whose function is efficiently and reproducibly to produce functional blood vessels has not been discovered yet and thus techniques for addressing such issues are required.

Easily accessible dental tissue is considered as the promising source of cell collection, including different types of mesenchymal stem cell (MSC)-like cells, dental pulp stem cells (DPSCs), stem cells from exfoliated deciduous teeth (SCEDs), stem cells from apical papilla (SCAPs), periodontal ligament stem cells (PDLSCs), and dental follicle progenitor cells (DFPCs). Dental stem/progenitor cells have typical fibroblast-like morphologies and MSC-like properties, and also can be reprogrammed into hiPSCs by using the four classical reprogramming factors (Oct4(O), Sox2(S), Klf4(K), and c-Myc(M), or Oct4, Sox2, Lin28(L), and Nanog(N)) (Journal of dental research, 89, 773-778, 2010). The OSKM factor combination is considered to be the most potent for providing consistent and reproducible hiPSC generation in a broad range of adult cell types, including dental cells. However, the use of the oncogenic factors Klf4 and c-Myc would greatly impede further clinical application. Therefore, the development of a reprogramming technique excluding or replacing oncogenic factors Klf4 and c-Myc is required.

Additionally, the lineage-specific differentiation potential of human dental pulp cell (hDPC)-derived hiPSCs (hDPC-iPSCs), particularly into EPCs, has not been demonstrated. Thus, the development of a safe and more efficient means of lineage-matched human dental cell reprogramming is needed.

### DISCLOSURE

### Technical Problem

Under these circumstances, the present inventors completed the present invention by examining the differentiation potential of hiPSCs, the treatment effects of ischemic disease and heart disease, and highly efficient methods for producing hDPC-iPSCs using only two reprogramming factors Oct4 and Sox2, excluding oncogenic factors Klf4 and c-Myc.

### Technical Solution

The purpose of the present invention is to provide a method for producing induced pluripotent stem cells from human dental pulp cells, comprising using only Oct4 and Sox2 as a reprogramming factor.

Another purpose of the present invention is to provide a method for producing endothelial progenitor cells, comprising differentiating induced pluripotent stem cells into endothelial progenitor cells, wherein the induced pluripotent stem cells are produced by the method for producing induced pluripotent stem cells.

Yet another purpose of the present invention is to provide a method for producing endothelial cells, comprising differentiating endothelial progenitor cells into endothelial cells, wherein the endothelial progenitor cells are produced by the method for producing endothelial progenitor cells.

Yet another purpose of the present invention is to provide a method for producing smooth muscle cells, comprising differentiating endothelial progenitor cells into smooth muscle cells, wherein the endothelial progenitor cells are produced by the method for producing endothelial progenitor cells.

Yet another purpose of the present invention is to provide the induced pluripotent stem cells having an enhanced rate of differentiation into endothelial progenitor cells, wherein the induced pluripotent stem cells are produced by the method for producing induced pluripotent stem cells.

Yet another purpose of the present invention is to provide the endothelial progenitor cells having an enhanced rate of differentiation into smooth muscle cells or endothelial cells, wherein the endothelial progenitor cells are produced by the method for producing endothelial progenitor cells.

Yet another purpose of the present invention is to provide the endothelial cells produced by the method for producing endothelial cells.

Yet another purpose of the present invention is to provide smooth muscle cells produced by the method for producing smooth muscle cells.

Yet another purpose of the present invention is to provide a method for producing a composition for treating ischemic disease, comprising:
(a) preparing induced pluripotent stem cells produced by the method of claim 1;
(b) preparing endothelial progenitor cells differentiated from the induced pluripotent stem cells;
(c) preparing endothelial cells differentiated from the endothelial progenitor cells; or
(d) preparing smooth muscle cells differentiated from the endothelial progenitor cells;
wherein the composition comprises induced pluripotent stem cells, endothelial progenitor cells, endothelial cells, smooth muscle cells, or a combination thereof, which are prepared in any one of steps (a) through (d).

Yet another purpose of the present invention is to provide a method for producing a composition for treating heart disease, comprising:
(a) preparing induced pluripotent stem cells produced by the method of claim 1;
(b) preparing endothelial progenitor cells differentiated from the induced pluripotent stem cells;
(c) preparing endothelial cells differentiated from the endothelial progenitor cells; or
(d) preparing smooth muscle cells differentiated from the endothelial progenitor cells;
wherein the composition comprises induced pluripotent stem cells, endothelial progenitor cells, endothelial cells, smooth muscle cells, or a combination thereof, which are prepared in any one of steps (a) through (d).

Yet another purpose of the present invention is to provide a method for treatment for ischemic disease comprising administering the composition produced by the method for producing the composition into an individual requiring the composition.

Yet another purpose of the present invention is to provide a method for treatment for heart disease comprising administering the composition produced by the method for producing the composition into an individual requiring the composition.

### Advantageous Effects

The present invention provides a highly efficient method for producing induced pluripotent stem cells from human dental pulp cells using only two non-oncogenic genes, Oct4 and Sox2, as a reprogramming factor. According to the present invention, a patient's dental pulp cells are an exceptional cell resource for producing induced pluripotent stem cells highly efficiently, compared to pre-presented tissue cell resources. Moreover, the present invention provides the optimized method for differentiation from a patient's dental pulp cell-derived induced pluripotent stem cells into endothelial progenitor cells, endothelial cells, and smooth muscle cells. Also, the present invention will make a significant contribution to treatment for ischemia disease and heart disease because a patient's dental pulp cell-derived differentiated cells can be used for cell treatment as a result of the cell treatment effect of the endothelial progenitor cells in the mouse models induced to have hind limb ischemia and myocardial infarction.

### DESCRIPTION OF DRAWINGS

FIG. 1a is the result of the typical morphology (original magnification, ×100) and Fluorescence-activated cell sorting (FACS) analysis using a fibroblast marker, 1B10, in cultured human dental pulp cells (hDPC) and human fibroblasts (hFib).

FIG. 1b is the result of schematic schedule of hiPSC generation and relative numbers of alkaline phosphatase (ALP)-positive colonies observed during 4F hFib-iPSC, 4F hDPC-iPSC, and 2F hDPC-iPSC generation.

FIG. 1c is the result of phase contrast, AP staining, and immunostaining of pluripotency markers (Nanog, SSEA3, SSEA4, Oct4, TRA1-81, and TRA1-60) of 2F hDPC-iPSCs. Counter-staining was performed with DAPI (original magnification, ×100).

FIG. 1d is the result of DNA methylation profiling of the Oct4 and Nanog promoters of hDPC and 2F hDPC-iPSC. To be more specific, the white and black circles represent unmethylated and methylated CpG dinucleotide respectively.

FIG. 1e is the result of the genomic integration of the exogenous factors (Oct4 and Sox2) in 2F hDPC-iPSC via genomic DNA PCR analysis.

FIG. 1f is the immunostaining result using antibodies for ectoderm (Tuji/Nestin), mesoderm (desmin/α-smooth muscle actin (α-SMA)), and endoderm (Foxa2 and Sox17) specific markers in embryoid bodies (EBs) differentiated from 2F hDPC-iPSCs at day 17. Counter-staining was performed with DAPI (original magnification, ×200).

FIG. 1g is the result of hematoxylin and eosin staining of 2F hDPC-iPSC-induced teratomas containing all three germ layers. To be more specific, ectoderm (neural rosette), mesoderm (cartilage), and endoderm (gut-like epithelium) were observed (original magnification, ×200).

FIG. 1h is the result of G-band karyotyping of 2F hDPC-iPSCs with normal karyotpe.

FIG. 2a is the result of phase contrast, ALP staining, and immunostaining of pluripotency markers (Nanog, SSEA3, SSEA4, Oct4, TRA181, and TRA1-60) of 4F hFib-iPSC. Counter-staining was performed with DAPI (original magnification, ×100).

FIG. 2b is the result of DNA methylation profiling of the Oct4 and Nanog promoters of hFib and 4F hFib-iPSC. To be more specific, the white and black circles represent unmethylated and methylated CpG dinucleotides, respectively.

FIG. 2c is immunostaining result using antibodies for ectoderm (Tuji/nestin), mesoderm (desmin/α-smooth muscle actin (α-SMA)), and endoderm (Foxa2 and Sox17) specific markers in EBs differentiated from 4F hFib-iPSC at day 17. Counterstaining was performed with DAPI (original magnification, ×200).

FIG. 2d is the result of the genomic integration of the exogenous factors (Oct4, Sox2, Klf4, and c-Myc) in 4F hFib-iPSC via genomic DNA PCR analysis.

FIG. 2e is the result of G-band karyotype of 4F hFib-iPSCs with normal karyotype.

FIG. 3a is the result of phase contrast, ALP staining, and immunostaining of pluripotency markers (Nanog, SSEA3, SSEA4, Oct4, TRA181, and TRA1-60) of 4F hDPC-iPSC. Counter-staining was performed with DAPI (original magnification, ×100).

FIG. 3b is the result of DNA methylation profiling of the Oct4 and Nanog promoters of hDPC and 4F hDPC-iPSC. To be more specific, the white and black circles represent unmethylated and methylated CpG dinucleotides, respectively.

FIG. 3c is immunostaining of result using antibodies for ectoderm (Tuj1/nestin), mesoderm (desmin/α-smooth muscle actin (α-SMA)), and endoderm (Foxa2 and Sox17) specific markers in EBs differentiated from 4F hDPC-iPSC at day 17. Counterstaining was performed with DAPI (original magnification, ×200).

FIG. 3d is the result of the genomic integration of the exogenous factors (Oct4, Sox2, Klf4, and c-Myc) in 4F hDPC-iPSC via genomic DNA PCR analysis.

FIG. 3e is the result of G-band karyotype of 4F hDPC-iPSC with normal karyotype.

FIG. 4a is the result of log₂ intensity plots of gene expression comparison between each cell line (4F hFib-iPSC, 4F hDPC-iPSC, 2F hDPC-iPSC, hFib, and hDPC) and H9 (H9 ESCs).

FIG. 4b is the result of heat map and clustering analysis of each cell line relative to H9 hESCs. To be more specific, genes displaying at least two-fold differential expression (24445 Genes) were selected. The scale ranges from 0.02 to 1. Red indicates log₂ intensity >1.7, and green indicates log₂ intensity <0.

FIG. 4c is the result of expression patterns of selected pluripotency-related genes in each cell line relative to H9 hESCs. The scale ranges from 0.05 to 1.

FIG. 5a is the result of log₂ intensity plots of gene expression comparison between each cell line (4F hFib-iPSC-EC(IMR-OSKM), 4F hDPC-iPSC-EC(DPC-OSKM), and 2F hDPC-iPSC-EC(DPC-OS)) and H9 hESC-ECs(ES).

FIG. 5b is the result of heat map and clustering analysis of each cell line relative to H9 hESC-derived ECs. To be more specific, genes displaying at least ten-fold differential expression were selected. The scale ranges from 0.79 to 1. Red indicates log₂ intensity >10, and green indicates log₂ intensity <0.

FIG. 5c is the result of expression patterns of selected pluripotency and endothelial-specific genes in each cell line relative to H9 hESCs (hES).

FIG. 6a is flow diagram of EPC generation through retroviral transduction using genes encoding OSKM(4F) or OS(2F).

FIG. 6b is the result which relative expression levels of mesoderm (T, MS×1, IGF2, and Runx2) and endothelial progenitor cell (CD34, CD31, VE-cad, and KDR) marker genes in 4F hFib-iPSC, 4F hDPC-iPSC, or 2F hDPC-iPSC were compared with hES at day 10. The values are presented as the means±SD of three independent experiments. The data shown in the graphs are expressed as the means±SD (n3). <0.01 and ***p<0.001 versus hES.

FIG. 6c is the result of FACS analysis of CD34 and CD31 co-expression in differentiated hiPSCs (4F hFib-iPSC, 4F hDPC-iPSC, and 2F hDPC-iPSC) at day 10.

FIG. 6d is the result of comparison of CD34⁺ cells in each differentiated hiPSCs (4F hFib-iPSC, 4F hDPC-iPSC, and 2F hDPC-iPSC) after MACS (magnet-activated cell sorting) at day 10.

FIG. 7a is the result of FACS analysis of the endothelial cell markers CD31 and CD105 in each hiPSC-derived ECs (4F hFib-iPSC-EC, 4F hDPC-iPSC-EC, and 2F hDPC-iPSC-EC).

FIG. 7b is the result of hiPSC-derived ECs (4F hFib-iPSC-EC, 4F hDPC-iPSC-EC, and 2F hDPC-iPSC-EC) showing typical endothelial cell morphology (A), and expressed multiple EC markers (vWF, CD31, and VE-cad) (B, C, and D).

FIG. 7c is the result of in vitro functional assay for hiPSC-derived ECs. ECs formed vascular tube-like structures on Matrigel (E) and absorbed Dil-labeled acetylated-LDL (F).

FIG. 8a is the result of morphologies and confocal images of SMC marker expression (α-SMA, elastin, calponin, and SM22) in hiPSC-derived SMCs (4F hFib-iPSC-SMC, 4F hDPC-iPSC-SMC, and 2F hDPC-iPSC-SMC).

FIG. 8b is the result of FACS analysis for the SMC markers calponin and SM22a in hiPSC-derived SMCs (4F hFib-iPSC-SMC, 4F hDPC-iPSC-SMC, and 2F hDPC-iPSC-SMC).

FIG. 9 is the result of contraction of hiPSC-derived SMCs (4F hFib-iPSC-SMC, 4F hDPC-iPSC-SMC, and 2F hDPC-iPSC-SMC) within 20 min after treatment with carbachol, and agonist against acetylcholine receptors.

FIG. 10a is the result of representative Matrigel plugs of each hiPSC-derived CD34⁺ cell group at day 7 after injection (A: control, B: 4F hFib-iPSC-CD34⁺ cells, C: 4F hDPC-iPSC-CD34⁺ cells, D: 2F hDPC-iPSC-CD34⁺ cells).

FIG. 10b is the graph of quantification of the hemoglobin concentrations in Matrigel plugs containing ((control cells (control), 4F hFib-iPSC-CD34⁺ cells (hFib-4FiPSC-CD34⁺), 4F hDPC-iPSC-CD34⁺ cells (hDPC-4FiPSC-CD34⁺), and 2F hDPC-iPSC-CD34⁺ cells (hDPC-2FiPSC-CD34⁺)).

FIG. 10c is the result of response between species-specific CD31 antibodies and Matrigel plugs. To be more specific, species-specific CD31 antibodies were used in order to discriminate mouse and human blood vessels. Mouse-specific CD31 antibodies are indicated as green and human-specific CD31 antibodies are indicated as red colors, respectively. Bar is 20 μm (A: control, B: 4F hFib-iPSC-CD34⁺ cells, C: 4F hDPC-iPSC-CD34⁺ cells, D: 2F hDPC-iPSC-CD34⁺ cells).

FIG. 11a is the result of immunostaining of Matrigel plugs containing control cells (a), 4F hFib-iPSC-CD34⁺ cells (b), 4F hDPC-iPSC-CD34⁺ cells (c), and 2F hDPC-iPSC-CD34⁺ cells (d) with mouse CD31 antibody

FIG. 11b is the graph of the number of mouse CD31⁺ cells in Matrigel plugs.

FIG. 12 is the result of treatment effects of hiPSC-derived CD34⁺ cells (4F hFib-iPSC-CD34⁻, 4F hDPC-iPSC-CD34⁺ and 2F hDPC-iPSC-CD34⁺) in mouse model of hind-limb ischemia.

FIG. 12a is the graph of the perfusion rate improvement of each group in ischemic mouse model.

FIG. 12b is the bar graph showing the necrosis ratio of each group in ischemic mouse model.

FIG. 12c is the result of treatment effects of hiPSC-derived CD34⁺ cells in mouse model of myocardial infarction (MI). The representative images taken from a Masson's trichrome-stained section. Muscle is stained in red, and collagen is stained in blue. MI (WT: control, MI: myocardial infarction, MI+cell: administrating 2F hDPC-iPSC-CD34⁺ cells into myocardial infarction).

FIG. 12d is the result of quantification of fibrosis in left ventricular (LV) in MI model.

FIG. 12e is the result of microvessel co-staining using mouse CD31 and human NA antibodies in MI model.

FIG. 12f is the result of the microvessel density estimated through CD31 antibody staining in MI model.

FIG. 12g is the result of TUNEL assay performed to assess the level of apoptosis in cardiac muscle tissues in MI model.

FIG. 12h is the graph of apoptopic index (AI) in MI model.

FIG. 13a is the result of the perfusion rate difference of each group in mouse model of hind-limb ischemia.

FIG. 13b is the result of coexistence of human nuclear antigen (hNA) and mouse CD31⁺ cells around operated region on POD 7 in mouse model of hind-limb ischemia.

FIG. 14 is the result of co-staining of microvessel tissues with mouse CD31 and human NA antibodies in MI model.

### BEST MODE

As one of the solutions for the above problems, the present invention provides a method for producing induced pluripotent stem cells (iPSCs) from human dental pulp cells (hDPCs) using only Oct4 and Sox2 as a reprogramming factor. Particularly, the present invention provides a method for (a) delivering genes of reprogramming factors, Oct4 and Sox2 to hDPCs; (b) culturing the cells of (a); and (c) purifying induced pluripotent stem cells from the cultured cells.

The term “reprogramming” in the present invention refers to the process of restoring the differentiation potential of differentiated cells. In addition, the meaning of the “reprogramming” is the same as the meaning of “dedifferentiation” and “cellular reprogramming”.

The term “induced pluripotent stem cells (iPSCs)” in the present invention refers to cells which are induced to have pluripotency by artificial reprogramming from differentiated cells. Artificial reprogramming can include usage of virus vectors made of retrovirus and lentivirus, or usage of nonvirus vectors, can be performed by taking nonvirus-meditated reprogramming factors using DNAs, RNAs, proteins, cell extracts, and/or small molecules, can include reprogramming processes performed by their combination and so on, but is not restricted thereto. The iPSCs have properties similar to embryonic stem cells (ESCs), specifically, having very similar cell shapes and gene and protein expression patterns, are pluripotent in-vitro and in-vivo, form teratoma, form chimeric mice when injected into mice blastocyst, and have a germline transmission ability.

The term “embryonic stem cells (ESCs)” in the present invention refers to self-renewal cells having pluripotentcy, which enables differentiation into all cell types of all tissues of an individual, and are cultured outside the body by extracting inner cell mass from a blastocyst just before a fertilized egg is implanted in the mother's fetus. Also, ESCs include embryonic stem cell-derived embryonic bodies (EBs) in a broad sense. The ESCs of the present invention can be ESCs from all sources such as human, monkey, pig, horse, cattle, sheep, dog, cat, mouse, rabbit and so on, but are preferably human ESCs (hESCs).

Moreover, the present invention can be a method for producing iPSCs, wherein nicotinamide is added to said culturing in step (b). The term “nicotinamide” in the present invention refers to one of several complex of water-soluble vitamins and vitamin B and is the amide of nicotinic acid. Nicotinamide exists in the body as a type of coenzyme such as nicotinamide nucleotide, NAD⁺ and NADP⁺ as well as takes part in lots of oxidation-reduction reactions as a coenzyme. Nicotinamide is used as a medicine for chronic alcoholism, angina and frostbite. Nicotinamide is contained in the liver, fish, grain embryos, yeast, soybean milk and meat. Nicotinamide is a colorless crystalline powder, is produced from tryptophan with nicotinic acid. Pellagra, a nicotinamide deficiency disease, causes skin dermatitis, diarrhea, abalienation, unrest and so on, even results in death. Nicotinamide causes stomach ulcer, diabetes and hepatotoxicity if the nicotinamide is consumed excessively.

An exemplary embodiment of the present invention shows that as a result of hDPCs in which reprogramming factors Oct4 and Sox2 are delivered being cultured on modified hESC medium (DMEM/F12 supplemented with 20% KnockOut Serum Replacement (KSR, Invitrogen), 1% NEAA (non-essential amino acids), 1 mM L-glutamine, 0.1 mM β-mercaptoethanol, 100 mg/ml of streptomycin, 100 U/ml of penicillin, and 10 ng/ml of basic fibroblast growth factor (bFGF, Invitrogen) after adding nicotinamide 1 mM, hDPCs using two reprogramming factors, OS(Oct4 and Sox2; 2F) were found to have more efficient pluripotency due to reprogramming than human lung fibroblasts (hFibs) using four OSKM reprogramming factors, (Oct4, Sox2, Klf4 and c-Myc; 4F).

Additionally, the present invention can provide a method for producing iPSCs, where step (a), step (b), and step (c) are simultaneously or sequentially performed, or step (b) and step (c) are repeatedly performed.

As another aspect, the present invention provides a method for producing endothelial progenitor cells (EPCs), comprising differentiating an iPSCs into EPCs, wherein the iPSCs is produced by the method. The EPCs can be blood progenitor cells. Particularly, the present invention provides a method for producing EPCs, comprising differentiating iPSCs into EPCs, wherein the iPSCs is produced by the method. Moreover, the present invention can be a method for producing EPCs, wherein the differentiating step comprises culturing iPSCs in a medium comprising BMP4 (bone morphogenetic protein 4), Activin A, VEGF-A (vascular endothelial growth factor-A) and bFGF.

The term, “endothelial progenitor cells (EPCs)” in the present invention refers to cells which are able to differentiate into endothelial cells (ECs) of blood vessels and lymphatic vessels. EPCs can have an ability of vasculogenesis and contribute to pathologic angiogenesis discovered in tumor. Preferably EPCs can be vascular progenitor cells.

An exemplary embodiment of the present invention shows that for CD34⁺ EPC differentiation from hiPSCs, the hiPSCs were sequentially stimulated using combinations of BMP4, Activin A, VEGF-A, and bFGF. Consequently, endoderm progenitor (CD34, CD31, VE-cadherin and KDR) markers were found to be expressed plentifully in the differentiated cells, and the ratio of CD34⁺/CD31⁺ cells is higher in cell populations differentiated from 2F hDPC-iPSCs (20.4%) than cell populations differentiated from 4F hFib-iPSCs (11.5%) and cell populations differentiated from 4F hDPC-iPSCs (10.3%). These results show that hDPC-hiPSCs have a strong preference for differentiation into CD34⁺ cells, and particularly, 2F hDPC-iPSCs have a better ability of CD34⁺ cell differentiation than 4F hDPC-iPSCs.

The term, “CD34” in the present invention refers to a cluster of differentiation 34, and is used as a marker for EPCs. CD34⁺ cells in the present invention refer to cells reacting positively to CD34, and the cells can be EPCs.

As another aspect, the present invention provides a method for producing ECs, comprising differentiating EPCs into ECs, wherein the EPCs are produced by the method for producing of EPCs. Particularly, the present invention can be a method for producing ECs, wherein said differentiating comprises culturing EPCs in a medium comprising VEGF-A and bFGF.

The term, “endothelial cells (ECs)” in the present invention refers to 1-layered squamous cells covering inner walls of blood vessels and lymphatic vessels. Preferably, ECs can refer to vascular ECs covering inner walls of blood vessels.

An exemplary embodiment of the present invention shows that highly purified CD34⁺ cells (>91% purity) were plated in 0.1% gelatin-coated plates (5×10⁴ cells per well in a 12-well plate) and cultured in EBM-2/EGM-2 (Lonza, Walkersville, Md., USA) supplemented with bFGF (50 ng/ml) and VEGF-A (100 ng/ml) for approximately 20 days. Consequently, the cells (2F hDPC-iPSC-ECs) differentiated from 2F hDPC-iPSC-derived CD34⁺ cells expressed typical endothelial markers, such as CD31 (99.1%) and CD105 (98.4%), at higher levels compared with those from 4F hFib-iPSC-derived CD34⁺ cells (CD31 (89.5%) and CD105 (87.1%)) and 4F hDPC-iPSC-CD34⁺ cells (CD31 (78.7%) and CD105 (76.2%)). Thus 2F hDPC-iPSC-CD34⁺ cells have high efficiency of differentiation into ECs, and are more favorable to apply to clinical trials because of the use of non-oncogenic genes Oct4 and Sox2.

An exemplary embodiment of the present invention shows that, to evaluate the ability of hDPC-iPSC-CD34⁺ cells in angiogenesis in vivo, a Matrigel implant assay was employed in mice. The quantification of hemoglobin in the Matrigel plugs from mice consistently indicated that hDPC-iPSC-CD34⁺ cells significantly increased angiogenesis. Moreover, the result from immunohistochemistry analysis of the Matrigel shows that the incorporation between human and mouse vascular cells was observed in the hDPC-iPSC-CD34⁺ cell transplants, showing that hDPC-iPSC-CD34⁺ cells could differentiate into ECs and cooperate with native ECs in vivo.

Additionally, the CD34⁺ cells derived from 2F hDPC-iPSCs exhibited higher angiogenic activity than those derived from 4F hDPC-iPSCs and 4F hFib-iPSCs.

As another aspect, the present invention provides a method for producing SMCs, comprising differentiating EPCs into SMCs, wherein the EPCs are produced by said method. Particularly, the present invention can be a method for producing SMCs, wherein said differentiating comprises culturing EPCs in a medium comprising bFGF and PDGF-BB (platelet-derived growth factor-BB).

The term “smooth muscle cells (SMCs)” in the present invention refers to cells comprising smooth muscle, and the smooth muscle is called non-striated muscle and is a counterpart of striated muscle. All visceral muscles except heart muscle in a vertebrate are non-striated muscles. SMCs are thin and have a spindle-like shape. SMCs typically have an oval core at the center but rarely have multiple cores. The contracting material is actomyosin and smooth muscles have few quantities of actomyosin compared to that in striated muscles, and they are developed in parts in which there is little activity. Smooth muscles have a slow contraction rate but it is an involuntary muscle which does not become exhausted easily. Non-striated muscles are under double control (sympathetic nerves and parasympathetic nerves) from the autonomic nervous system. To be more specific, there are muscles strongly governed by nerves such as nictitating membranes and blood vessel muscles, and there are muscles weakly governed by nerves and thus autonomously contract, such as uterus muscles and intestine muscles. Non-striated muscles have more remarkable humoral control than striated muscles, as it appears that oxytocin can be administrated in the uterus, causing the uterus to contact.

An exemplary embodiment of the present invention shows that, as a result of culturing EPCs in EBM-2/EGM-2 (Lonza) supplemented with bFGF and PDGF-BB (Invitrogen), the EPCs are examined to differentiate into SMCs. Calponin⁺ (94.8%) and SM22⁺ (80.6%) SMCs were notably higher in cell populations of SMCs (2F hDPC-iPSC-SMCs) differentiated from 2F hDPC-iPSC-CD34⁺ cells compared with those from 4F hFib-iPSCs-CD34⁺ (calponin (87.7%) and SM22a (65.6%)) and 4F hDPC-iPSCs-CD34⁺ (calponin (79.2%) and SM22a (53.9%)). Thus 2F hDPC-iPSC-CD34⁺ cells have high efficiency of differentiation into SMCs, and are more favorable to apply to clinical trials because of using non-oncogenic genes Oct4 and Sox2.

As another aspect, the present invention provides iPSCs having an enhanced rate of differentiation into EPCs, wherein the iPSCs are produced by the method for producing iPSCs; EPCs having an enhanced rate of differentiation into SMCs or ECs, wherein the EPCs are produced by the method for producing EPCs; ECs produced by the method for producing ECs; and SMCs produced by the method for producing SMCs.

Preferably, iPSCs having an enhanced rate of differentiation into EPCs, wherein the iPSCs are produced by the method comprising:
(a) delivering genes of Oct4 and Sox2 into human dental pulp cells;
(b) culturing the cells and adding nicotinamide; and
(c) purifying iPSCs from the cultured cells.

The “iPSCs”, “EPCs”, “ECs”, and “SMCs” in the present invention are the same as described above.

As another aspect, the present invention provides a composition for treating ischemic disease and/or heart disease, comprising more than 1 of iPSCs having an enhanced rate of differentiation into EPCs, wherein the iPSCs are produced by the method for producing iPSCs; EPCs having an enhanced rate of differentiation into SMCs or ECs, wherein the EPCs are produced by the method for producing EPCs; ECs produced by the method for producing ECs; and SMCs produced by the method for producing SMCs, as an effective component. Particularly, the present invention can provide a composition for treating ischemic disease and/or heart disease, wherein the ischemic disease can be hind-limb ischemia and heart disease can be myocardial infarction.

The “iPSCs”, “EPCs”, “ECs”, and “SMCs” in the present invention are the same as described above.

As another aspect, the present invention provides a method for producing a composition for treating ischemic disease, comprising:
(a) preparing iPSCs produced by the method for producing of iPSCs;
(b) preparing EPCs differentiated from the iPSCs;
(c) preparing ECs differentiated from the EPCs; or
(d) preparing SMCs differentiated from the EPCs;
wherein the composition comprises iPSCs, EPCs, ECs, SMCs, or a combination thereof, which are prepared in any one of steps (a) through (d).

Particularly, the ischemic disease can be hind-limb ischemia.

The “iPSCs”, “EPCs”, “ECs”, and “SMCs” in the present invention are the same as described above.

As another aspect, the present invention provides a method for producing a composition for treating heart disease, comprising:
(a) preparing iPSCs produced by the method for producing of iPSCs;
(b) preparing EPCs differentiated from iPSCs;
(c) preparing ECs differentiated from the EPCs; or
(d) preparing SMCs differentiated from the EPCs;
wherein the composition comprises iPSCs, EPCs, ECs, SMCs, or a combination thereof, which are prepared in any one of steps (a) through (d).

Particularly, heart disease can be myocardial infarction.

The “iPSCs”, “EPCs”, “ECs”, and “SMCs” in the present invention are the same as described above.

The term “ischemic disease” in the present invention indicates a disease which arises because blood supply is insufficient, in part resulting from various causes such as angiostenosis or vasoconstriction, thrombosis or embolus.

Examples of ischemic disease are congestive heart failure, hypertensive heart disease, arrhythmia, congenital heart disease, myocardial infarction, stroke, peripheral vascular disease, angina pectoris, cerebrovascular dementia, coronary artery imperfection, brain dysfunction, vascular imperfection, and hind-limb ischemia, and so on, but not limited thereto. Additionally, all ischemic diseases for which angiogenesis is required can be included but not limited thereto. Preferably, the ischemic disease can be hind-limb ischemia.

The term “heart disease” in the present invention indicates a disease which involves the heart, the blood vessels or both. Preferably, heart disease can be myocardial infarction.

The term “treating” in the present invention refers to all activities making the symptoms of ischemia and heart attack better or beneficially altered by transplantation of the induced pluripotent stem cells, endothelial progenitor cells, ECs, SMCs, or a combination thereof.

Said composition can be well-known to those skilled in the art, or comprise materials used generally, helping storage, implantation and settlement of living iPSCs, EPCs, ECs, SMCs, or a combination thereof. In other words, the composition can comprise a matrix physiologically compatible to cells, or diluting agents physiologically compatible to cells. The type of matrix and/or diluting agents can be selected by those skilled in the art, depending on intended administration routes. The composition can comprise selectively active elements, or other acceptable diluting agents used when cell treatment is performed.

Said composition can comprise an appropriate level of iPSCs, EPCs, ECs, SMCs, or a combination thereof. Particularly, the iPSCs can be 10⁵ to 10⁷ cells/cycle, the EPCs be 10⁶ to 10⁹, the ECs be 10⁶ to 10⁹, SMCs be 10⁶ to 10⁹. To be more detailed, the iPSCs can be 10⁵ to 10⁶ cells/cycle, the EPCs be 10⁶ to 10⁷, the ECs be 10⁶ to 10⁷, SMCs be 10⁶ to 10⁷.

As another aspect, the present invention provides a method of treatment for ischemic disease, comprising administering the composition produced by the method for producing a composition for treating ischemic disease, to an individual requiring the composition. Preferably, the ischemic disease can be hind-limb ischemia.

As another aspect, the present invention provides a method of treatment for heart disease, comprising administering said composition produced by the method for producing a composition for treating heart disease, to an individual requiring the composition. Preferably, the heart disease can be myocardial infarction.

“Ischemic disease”, “heart disease” and “treating” in the present invention are the same as described above.

“An individual” in the present invention refers to all animals including human who have or could contract the ischemic disease, such as monkey, cattle, horses, sheep, pigs, chicken, turkey, quails, cats, dogs, mice, rabbits, or guinea-pigs. Additionally, the composition of the present invention can treat or prevent the disease effectively by administration of the composition to the individual. The composition of the present invention can be administrated with pre-existing medicines.

“Administrating” in the present invention refers providing materials for a patient in a desirable and arbitrary way, and the administration route of the composition can be any general route as long as it arrives to the target tissues. Examples of the administration routes are intraperitoneal administration, intravenous administration, intramuscular administration, subcutaneous administration, intradermal administration, oral administration, topical administration, intranasal administration, intrapulmonary administration, and rectal administration, but are not limited thereto. Moreover, the pharmaceutical composition can be administrated by an arbitrary device which can make active materials move into target cells. The administration composition can be manufactured by using soluble solvents like physiological saline and Ringer's solution, and by using non-soluble solvents like plant oils, high-ranked fatty acid ester (e.g. ethyl oleate), and alcohols (e.g. ethanol, benzyl alcohol, propylene glycol, glycerin). Moreover, the pharmaceutical composition can include a pharmaceutical carrier like stabilizers for prohibiting alteration (e.g. ascorbic acid, sodium bisulfite, sodium sulfite fatigue, BHA, tocopherols, EDTA), emulsifiers, buffers for pH control, and preservatives for prohibiting bacteria growth (e.g. phenyl mercury nitrate, chimerosal, benzalkonium chloride, phenol, cresol, benzyl alcohol).

The composition of the present invention can be administrated in pharmaceutically acceptable quantity.

The “pharmaceutically acceptable quantity” of the present invention refers to a desirable quantity to the extent that results in sufficient treatment of a disease at a rational ratio of benefit/risk for medical treatments, and does not result in side effects. The acceptable quantity depends on patient's health status, type of a disease, severity, medicine activity, medicine sensitivity, administration method, administration time, administration routes and ejection ratio, treatment period, elements used simultaneously or in combination, and other element well known in medical field. The composition of the present invention can be administrated as a single medicine, used jointly with other medicines, used simultaneously or sequentially with pre-existing medicines, or used in a single or multiple administrations. What is important is to administer a quantity of the medicine in the extent that results in the best effect with the least quantity, without side effects after considering all of the elements, and the quantity can be easily determined by those skilled in the art. Detailed dosage is described above.

In an exemplary embodiment of the present invention, CD34⁺ cells from each hiPSCs and DMEM, as a control, were injected intramuscularly in a hind limb ischemia-induced mouse, in order to examine cell treatment effect in mouse hind limb ischemia model. Time-series indocyanine green (ICG) perfusion imaging was performed immediately after surgery (day 0) and on post-operative days (POD) 3 and 7. Consequently, CD34⁺ cells from 2F hDPC-iPSCs were more effective in the restoration of perfusion in ischemic hind limbs and reducing limb necrosis, compared with those from 4F hDPC-iPSCs and 4F hFib-iPSCs.

Additionally, in order to determine whether transplanted hiPSC-CD34⁺ cells recover the ischemic hind limb, tissue samples were harvested from the operated region on POD 7. As a result, coexistence of human nuclear antigen (hNA) and mouse CD31⁺ cells was observed in ischemic hind-limb regions injected with hiPSC-CD34⁺ cells, suggesting that hiPSC-CD34⁺ cells develop into ECs in vivo, thereby contributing to neovasculogenesis in the ischemic region.

In an exemplary embodiment of the present invention, in order to assess the therapeutic effect of 2F hDPC-iPSC-CD34⁺ cells in a mouse model of acute myocardial infarction, hiPSC-CD34⁺ cells were administrated to a mouse induced myocardial infarction, particularly into three sites around the occluded region. One week after the induction of myocardial infarction and injection of CD34⁺ cells, the size of the left ventricular (LV) infarct was evaluated in both the transplanted and untransplanted groups. Consequently the administration of CD34⁺ cells significantly reduced the infarct size and the degree of fibrosis in the infarct zone compared with the controls. Additionally, as an analysis result of histology, 2F hDPC-iPSC-CD34⁺ cells effectively restored the density of CD31⁺ microvessels in the infracted myocardial tissues. As a result of TUNEL analysis, 2F hDPC-iPSC-CD34⁺ cells are involved, either directly or indirectly, in neovascularization, ameliorating myocardial infarction-induced cardiac dysfunction in mice.

2F hDPC-iPSCs, 2F hDPC-iPSC-EPCs, 2F hDPC-iPSC-ECs, or 2F hDPC-iPSC-SMCs of the present invention can be used as a medicine for ischemic disease because the fact that EPCs from 2F hDPC-iPSCs develop into ECs in vivo, thereby contributing to vasculogenesis and neovascularization in the ischemic region is examined.

### MODE FOR INVENTION

Hereinafter, the present invention will be described in detail through examples. However, the examples are only for illustration of the present invention and the present invention is not limited thereto.

### Example 1

### Cell Culture

1-1: Culture of Human Dental Pulp Cells (hDPCs) and Human Lung Fibroblasts (hFibs)

HDPCs were kindly provided by Prof. Joo-Cheol Park (Seoul National University, South Korea) and maintained in Dulbecco's modified Eagle's medium (DMEM, Invitrogen, Carlsbad, Calif., USA) supplemented with 100 U/ml of penicillin, 100 μg/ml of streptomycin (Invitrogen), and 10% fetal bovine serum (FBS, Invitrogen). Primary hFibs (IMR90, ATCC, Manassas, Va., USA) were cultured in MEM supplemented with 10% FBS, 100 μg/ml of streptomycin, 100 U/ml of penicillin, 1% non-essential amino acids (NEAA, Invitrogen), and 1 mM sodium pyruvate.

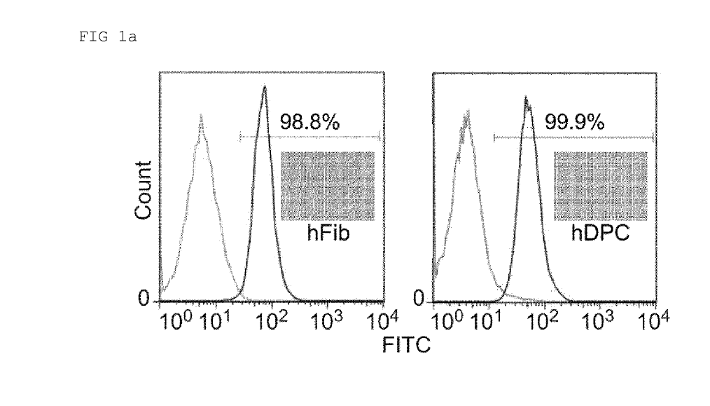

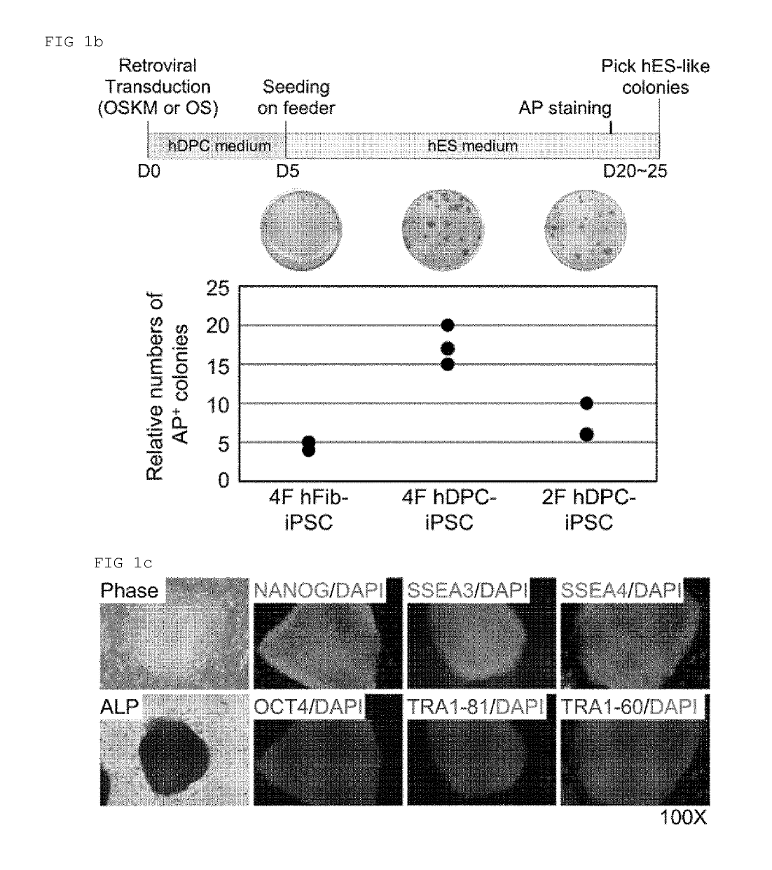

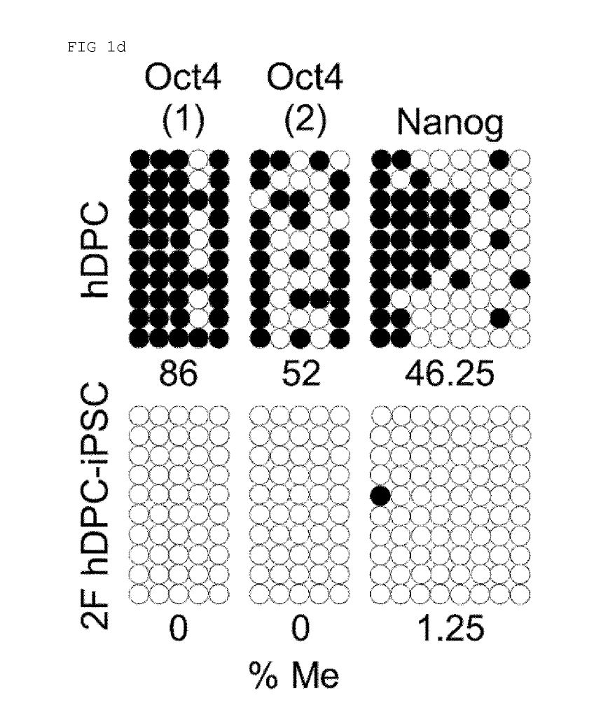

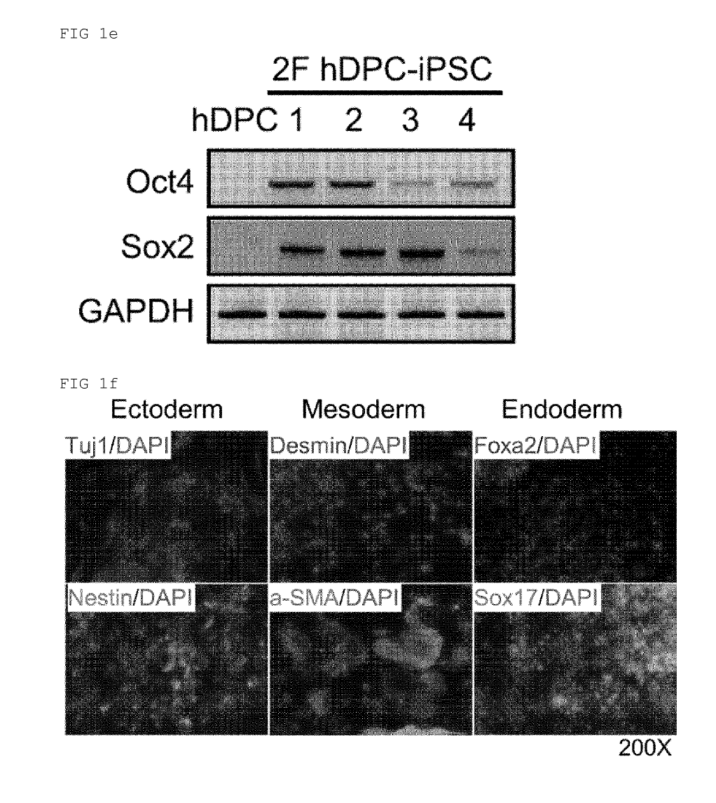

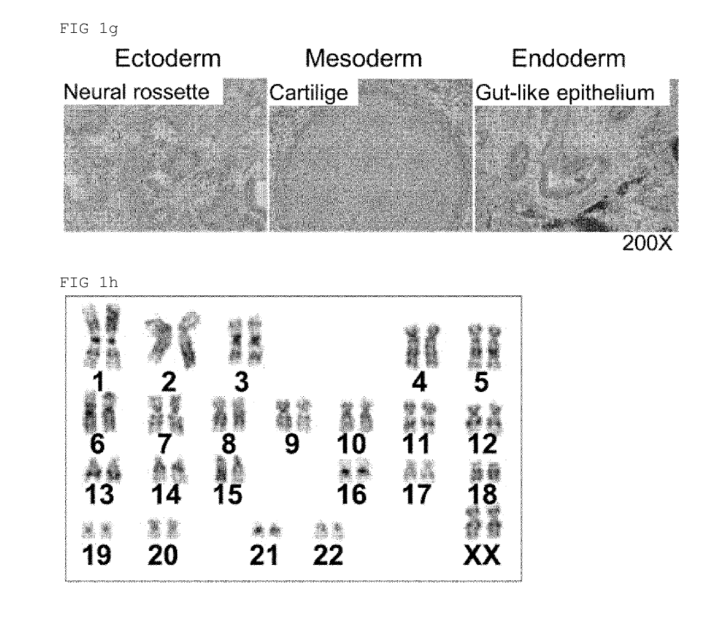

After serial culturing, almost all cells exhibited a homogeneous fibroblastic morphology and were positive for fibroblast-specific marker 1B10 (FIG. 1a).

1-2: Culture of H9 Human Embryonic Stem Cells (hESCs) and Human Induced Pluripotent Stem Cells (hiPSCs)

The H9 hESCs (WiCell Research Institute, Madison, Wis., USA) and hiPSCs were maintained on γ-irradiated mouse embryonic fibroblasts (MEFs) as feeder cells with hESC medium (DMEM/F12 supplemented with 20% KnockOut Serum Replacement (KSR, Invitrogen), 1% NEAA, 1 mM L-glutamine, 0.1 mM β-mercaptoethanol, 100 μg/ml of streptomycin, 100 U/ml of penicillin, and 10 ng/ml of basic fibroblast growth factor (bFGF, Invitrogen)). The feeder cells were plated on dishes coated with 0.1% gelatin (Sigma, St. Louis, Mo., USA). To maintain the hESCs and hiPSCs in an undifferentiated state, the medium was refreshed daily, and the cells were passaged once a week. The cells were maintained at 37° C. in 5% CO₂ in a high humidity environment.

### Example 2

### Production of hiPSCs

pMX-retroviral vectors coding for human Oct4, Sox2, Klf4, and c-Myc (OSKM, Addgene Inc., Cambridge, Mass., USA) and packaging vectors pCMV-VSVG were co-transfected into GP2-293 cells using calcium-phosphate mammalian transfection kit (Clontech, Palo Alto, Calif., USA). 48 h and 72 h after transfection, the virus-containing supernatants were collected, filtered (0.45-μm filter, Millipore, Billerica, Mass., USA) and concentrated through ultracentrifugation (Beckman, Palo Alto, Calif., USA). The GFP receptor virus was used in virus titration. For the hiPSC generation, hDPCs and hFibs were seeded at a density of 1×10⁵ cells per well in a six-well plate. Twenty four hours later, the cells were infected with virus and maintained with the respective primary cell culture medium in the presence of 8 μg/ml of polybrene (Sigma). Five days post-transduction, the cells were reseeded and further cultured in modified hESC medium after adding 1 mM nicotinamide. The medium was changed every other day until hESC-like colonies emerged. To establish hiPSC lines, the colonies were mechanically picked and transferred onto four-well dishes pre-seeded with MEF feeder cells. Each colony was further maintained and expanded for subsequent experiments.

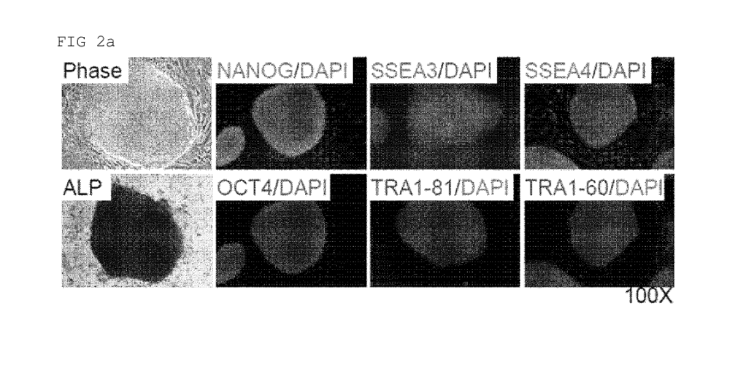

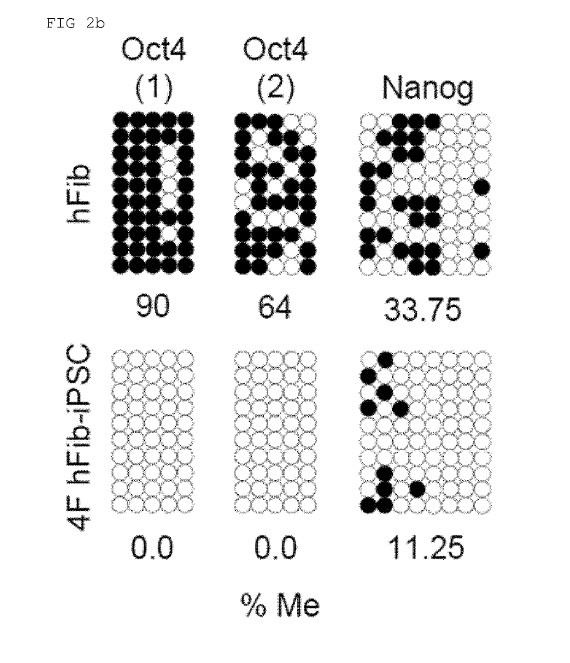

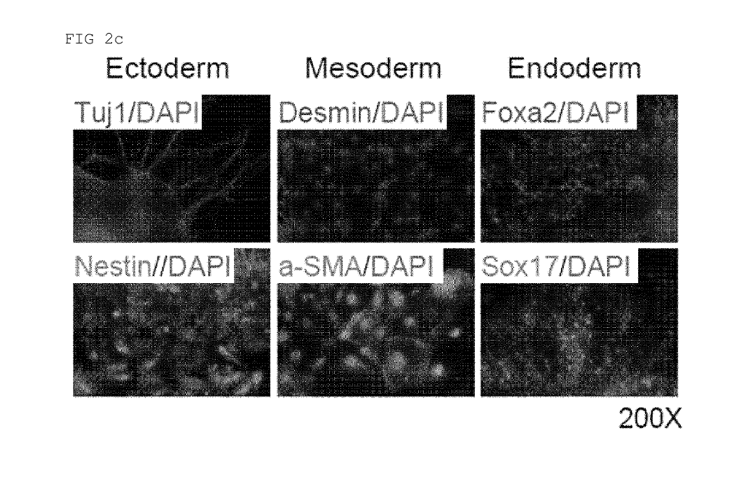

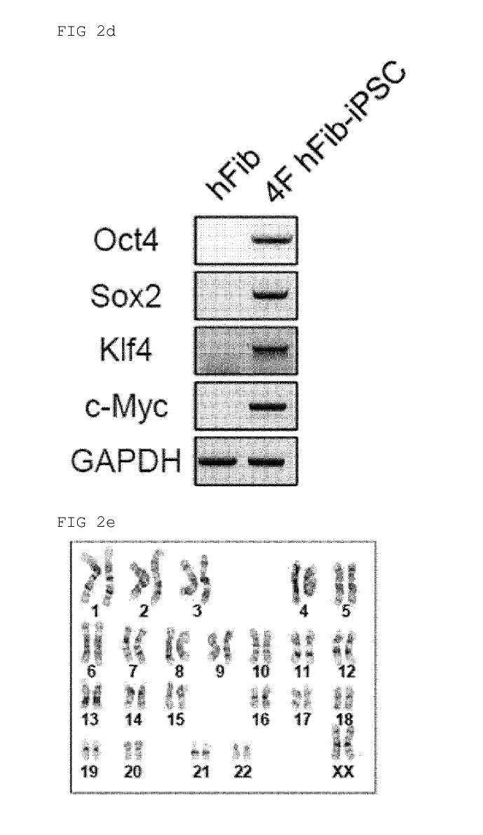

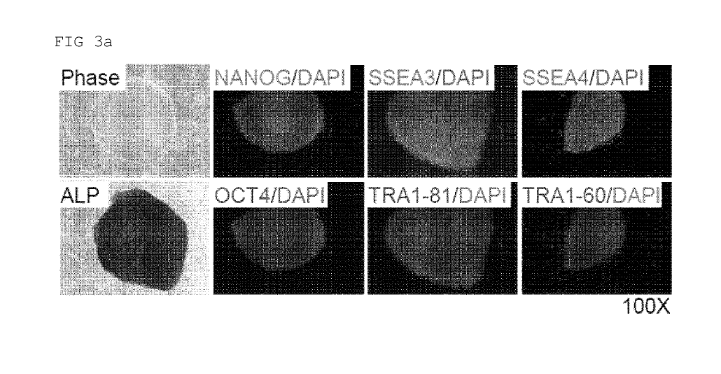

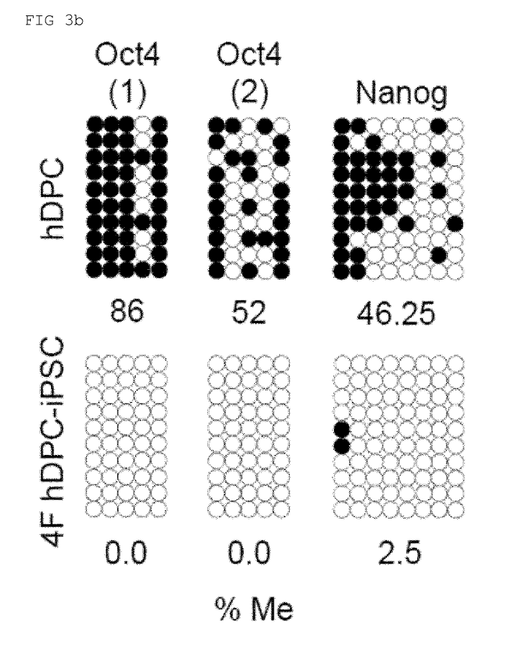

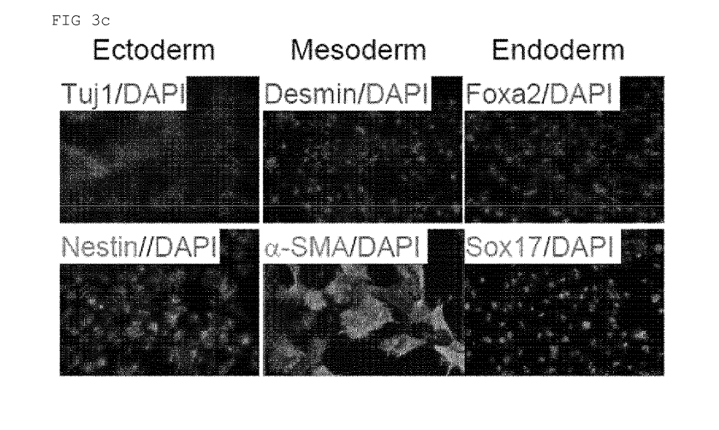

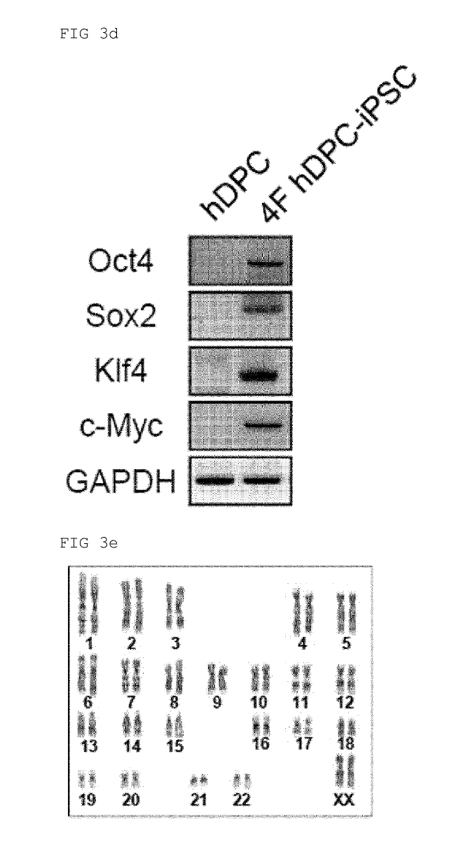

Additionally, hFibs-derived iPSCs generated by using OSKM(Oct4(O), Sox2(S), Klf4(K), and c-Myc(M); 4F) (4F hFib-iPSCs) (FIGS. 2a to 2e), hDPCs-derived iPSCs generated by using OS(2F hDPC-iPSCs) (FIGS. 1b to 1h), and hDPCs-derived iPSCs generated by using OSKM(4F hDPC-iPSCs) (FIGS. 3a to 3e) were selected based on their distinctive morphologies and properties equivalent to hESCs.

The alkaline phosphatase (AP)⁺ hESC-like colonies were counted to quantify the reprogramming efficiency (FIG. 1b). Consequently, the hDPCs through the ectopic expression of the two reprogramming factors OS were more efficiently reprogrammed to pluripotency than the reprogramming of hFibs with the ectopic expression of the four reprogramming factors OSKM.

Meanwhile, under the optimal reprogramming conditions used in the present invention, the hDPCs were successfully reprogrammed into hiPSCs using only two non-oncogenic factors, Oct4 and Sox2, whereas the hFibs were not fully reprogrammed using these two factors. The hESC-like colonies derived from the hDPCs emerged within three weeks after transduction, which is a week faster than the emergence of the hFibs. These results suggest that hDPCs have greater potential than hFibs in terms of hiPSC generation safety, efficiency, and kinetics.

### Example 3

### Characterization Analysis of hiPSCs

3-1: The Pre-Existing Analysis Method

AP staining, embryoid body (EB) formation, immunohistochemistry, genomic DNA PCR, bisulfite pyrosequencing, genotyping, karyotyping, and teratoma formation were performed through the pre-existing methods.

3-2: Microarray Analysis

Total RNAs were extracted using the RNeasy Mini Kit (Qiagen, Valencia, Calif., USA), labeled with Cy3, and hybridized to Agilent Whole Human Genome 4×44K Microarrays (two-color platform) according to the manufacturer's instructions. The hybridized images were scanned using an Agilent DNA microarray scanner and quantified with Feature Extraction software (Agilent Technology, Palo Alto, Calif., USA).

The normalization of the data and determination of the fold-change in gene expression were performed using GeneSpring GX7.3 (Agilent Technology). The average of the normalized signal channel intensity was divided by the average of the normalized control channel intensity to calculate the averages of the normalized ratios.

3-3: Results

Established 2F hDPC-iPSCs showed an enriched expression of pluripotency markers as determined using AP staining and immunohistochemistry (FIG. 1c) and displayed hypomethylation at the Oct4 and Nanog promoter regions (FIG. 1d). The transgenes used for reprogramming were all integrated into the genome of 2F hDPC-iPSCs (FIG. 1e). The pluripotency of hDPC-iPSCs was further determined by their in vitro differentiation potential into three germ layers, using antibodies for ectoderm (anti-Tuj1 and anti-nestin), mesoderm (anti-desmin and anti-α-SMA), and endoderm (anti-Foxa2 and anti-Sox17) (FIG. 1f). The histological analysis of the in vivo teratomas revealed that the 2F hDPC-iPSCs differentiates into various tissues representing all germ layers, including a neural rosette (ectoderm), cartilage (mesoderm), and gut-like epithelia (endoderm) (FIG. 1g). The 2F hDPC-iPSCs retained normal karyotypes after serial passages (FIG. 1h).

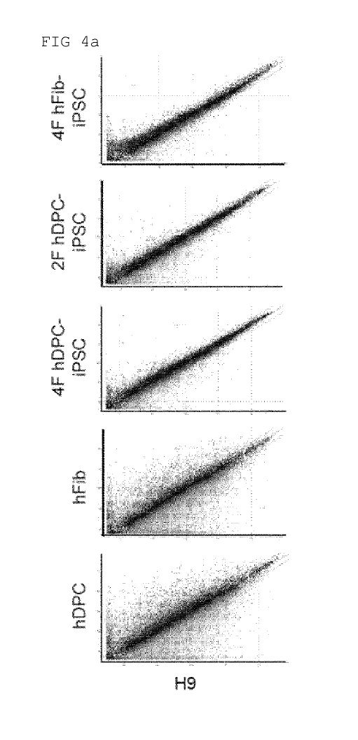

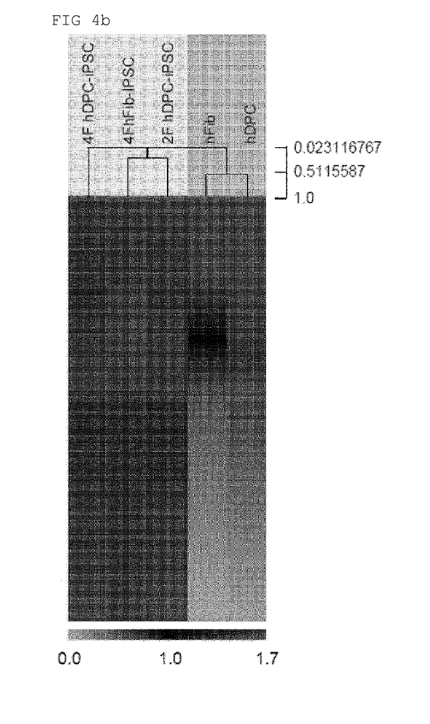

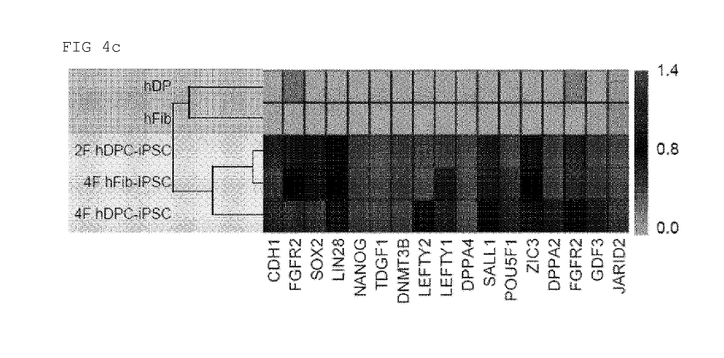

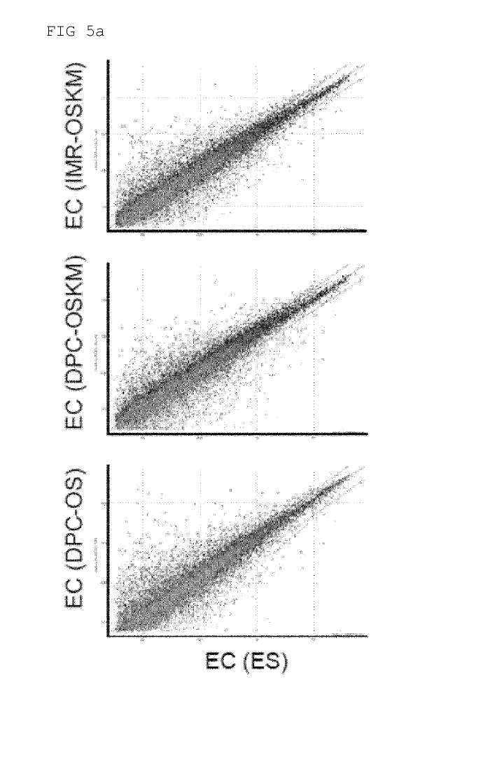

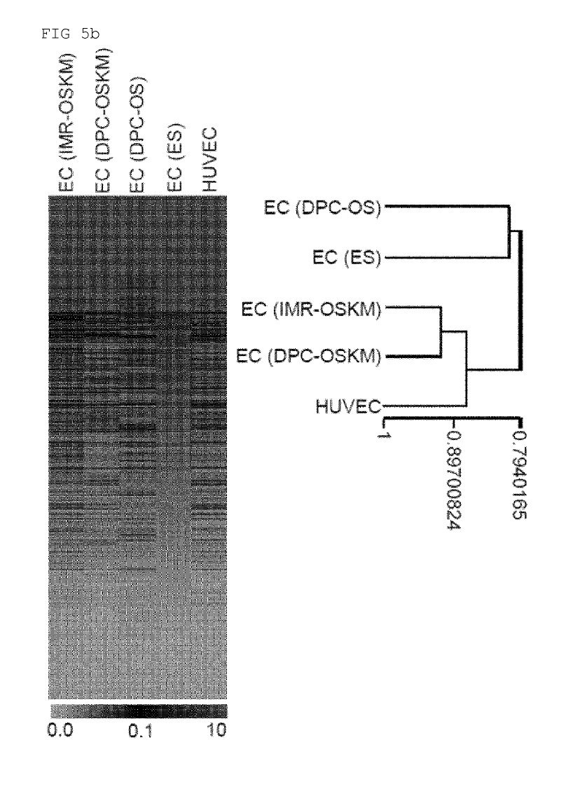

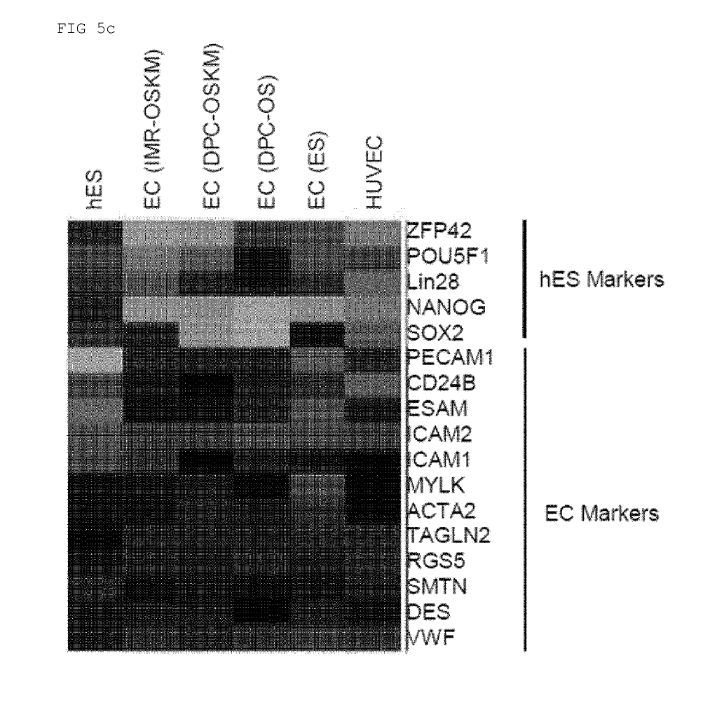

The global gene expression profiles obtained from hDPC-iPSCs were highly consistent with those obtained from H9 hESCs, as shown in scatter plots and a heat map (FIGS. 4a to 4c). Also, the global gene expression profiles obtained from hDPC-iPSCs-derived endothelial cells (hDPC-iPSC-ECs) were highly consistent with those obtained from H9 endothelial cells (H9 ECs) (FIGS. 5a to 5c). The established iPSCs maintained their undifferentiated states for more than 50 passages without the loss of self-renewal capacity and pluripotency.

### Example 4

### In Vitro Differentiation of hiPSCs

4-1: Differentiation of hDPC-hiPSCs into CD34⁺ Endothelial Progenitor Cells (EPCs)

For CD34⁺ EPCs differentiation, the hiPSCs were sequentially stimulated using combinations of BMP4 (bone morphogenetic protein 4), Activin A, VEGF-A (vascular endothelial growth factor-A), and bFGF as the pre-existing methods with slight modifications. Briefly, to form embryoid bodies (EBs) in suspension, monolayer cultured hiPSCs were harvested through treatment with 1 mg/ml of type IV collagenase and 500 μg/ml of dispase and transferred to Petri dishes containing hESC medium supplemented with 20 ng/ml of BMP4 (R&D Systems, Minneapolis, Minn., USA) and 10 ng/ml Activin A (R&D Systems). On day 3, the EBs were attached onto Matrigel-coated culture dishes and further differentiated using medium containing 100 ng/ml of VEGF-A (Invitrogen) and 50 ng/ml of bFGF (R&D Systems) for an additional 7 days. On day 10, differentiated cells were single-cell dissociated using 0.25% trypsin/EDTA (Invitrogen), and CD34⁺ cells were purified through magnet-activated cell sorting (MACS) (Miltenyi Biotech, San Diego, Calif., USA) using a CD34 antibody according to the manufacturer's instructions.

4-2: Further Differentiation of EPCs into ECs

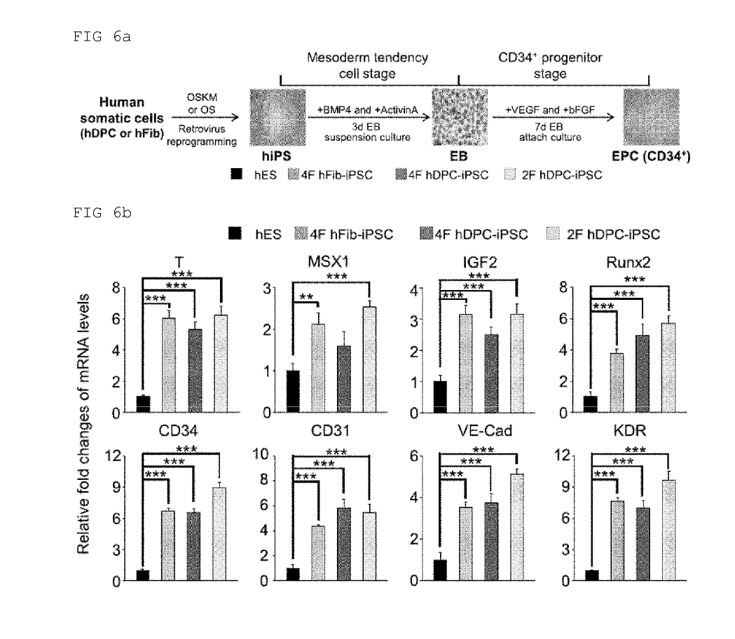

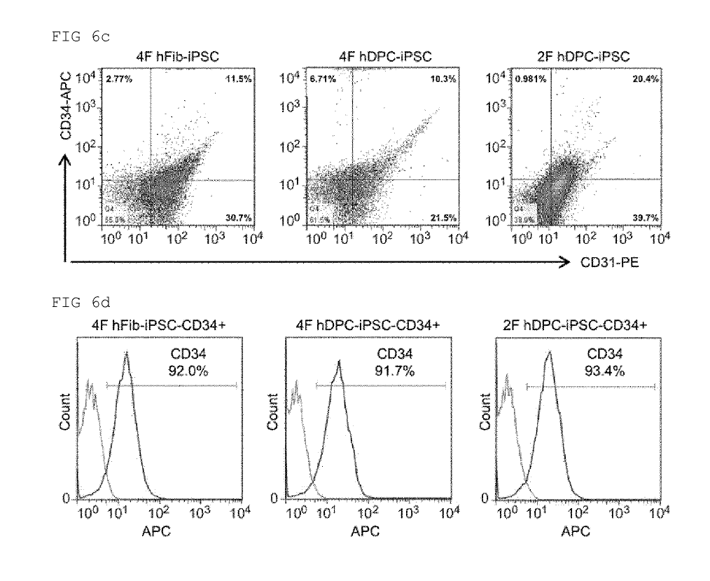

In order to assess the pluripotency of EPCs, the differentiation potential of CD34⁺ cells into ECs was examined. For differentiation into ECs, highly purified CD34⁺ cells (>purity 91%) isolated from differentiated cell populations from 4F hFib-hiPSCs, 4F hDPC-hiPSCs, and 2F hDPC-iPSCs using MACS (FIG. 6d) were plated in 0.1% gelatin coated plates (5×10⁴ cells per well in a 12-well plate) and cultured in EBM-2/EGM-2 (Lonza, Walkersville, Md., USA) supplemented with bFGF (50 ng/ml) and VEGF-A (100 ng/ml) for approximately 20 days.

4-3: Further Differentiation of EPCs into SMCs

In order to assess the pluripotency of EPCs, the differentiation potential of CD34⁺ cells into smooth muscle cells (SMCs) was examined. For SMCs differentiation, the purified CD34⁺ cells were plated in 0.1% gelatin-coated plates (5×10⁴ cells per well in a 12-well plate) and cultured in EBM-2/EGM-2 (Lonza) supplemented with 50 ng/ml of bFGF and 50 ng/ml of platelet-derived growth factor-BB (PDGF-BB, Invitrogen) for approximately 20 days.

4-4: Flow Cytometry Analysis

The cells differentiated from hiPSCs were harvested using 0.25% trypsin/EDTA for 2-3 min at 37° C. and resuspended in PBS/1% FBS at 1×10⁶ cells per 100 μl. The cells were labeled with antibodies against CD105-FITC, CD31-PE, and CD34-APC (all from Miltenyi Biotech), calponin (Sigma), and SM22a (Abcam, Inc., Cambridge, Mass., USA) at 4° C. for 30 min, washed twice with PBS/1% FBS, and analyzed using a FACSCanto II flow cytometer (BD Biosciences, Bedford, Mass., USA) according to the manufacturer's instructions. The data were analyzed using BD FACSDiva (BD Biosciences).

4-5: Immunofluorescence Staining

The cells differentiated from hiPSCs were washed with PBS and fixed in 4% formaldehyde at room temperature (RT) for 15 min. The cells were permeabilized in 0.1% Triton X-100 in PBS and blocked with blocking solution (4% bovine serum albumin (BSA)) for 1 h at RT. Primary antibodies against CD31/PECAM-1 (Abcam, 1:100), von Willebrand factor (vWF) (Abcam, 1:500), VE-cadherin (Abcam, 1:500), α-smooth muscle actin (α-SMA; Sigma, 1:500), elastin (Abcam, 1:1000), and calponin (Sigma, 1:1000) were diluted using blocking solution and incubated with the prepared cells overnight at 4° C. After washing three times with phosphate-buffered saline containing 0.01% Tween-20 (PBST; Invitrogen), the cells were incubated with the Alexa Fluor 488- or 594-conjugated secondary antibodies (Invitrogen) for 45 min at RT in the dark. After washing three times with PBST, the cells were incubated with 300 ng/ml of 40,6-diamidino-2-phenylindole (DAPI; Sigma) for 10 min to counterstain the cell nuclei.

4-6: Vascular Tube-Like Formation and Acetylated Low-Density Lipoprotein (LDL) Uptake

A total of 250 ml of growth factor-reduced Matrigel (10 mg protein/ml) was pipetted into 12-well tissue culture plates and polymerized for 30 min at 37° C. hiPSC-derived ECs (hiPSC-ECs) were incubated on Matrigel in EGM-2 supplemented with 10 μg/ml of VEGF-A. After 20 h, the vascular tube-like structures were observed using an inverted microscope. For the low-density lipoprotein (LDL) uptake assay, hiPSC-ECs were incubated with μg/ml of 1,1′-dioctadecyl-3,3,3′,3′-tetramethylindocarbocyanine (Dil)-labeled acetylated LDL (Invitrogen) for 5 h. The red fluorescent signals were detected using an Axiovert 200M fluorescence microscope (Carl Zeiss, Gottingen, Germany).

4-7: Results

(1) Established hDPC-iPSCs and hFib-iPSCs were assessed for lineage-specific differentiation potential into CD34⁺ EPCs, as depicted in FIG. 6a. After treatment of BMP4/Activin A for 3 days and VEGF-A/bFGF for another 7 days, the differentiated cells abundantly expressed mesodermal (Brachyury(T), MS×1, IGF2 and Runx2) and endothelial progenitor (CD34, CD31, VE-cadherin, and KDR) markers, as confirmed through qPCR (FIG. 6b). The flow cytometry analysis revealed that the ratio of CD34⁺/CD31⁺ cells was relatively higher in differentiated cell populations from 2F hDPC-iPSCs (20.4%) than those from 4F hFib-iPSCs (11.5%) and 4F hDPC-iPSCs (10.3%) (FIG. 6c).

These results suggest that hiPSCs derived from hDPCs have a strong preference for differentiation into CD34⁺ cells, and notably, two factor (2F)-induced hDPC-iPSCs displayed a more prominent ability for CD34⁺ cell differentiation than 4F hDPC-iPSCs.

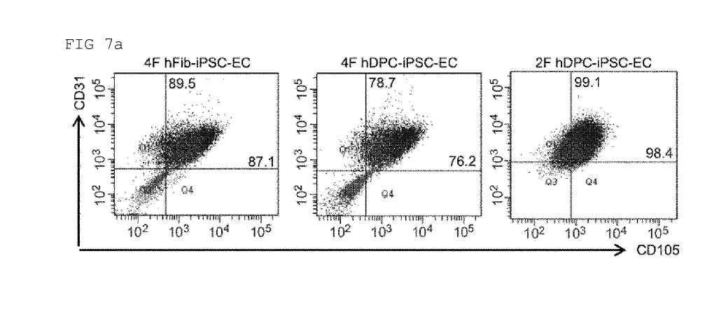

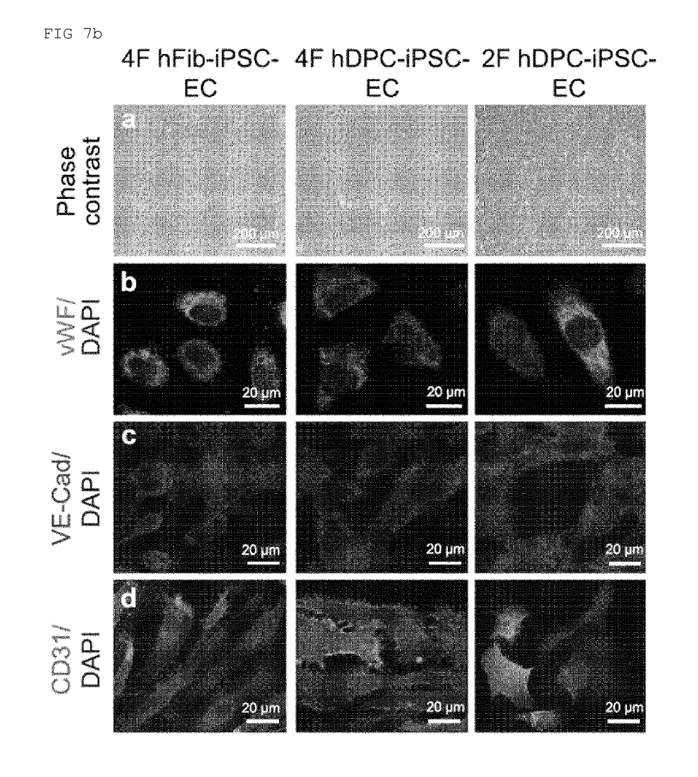

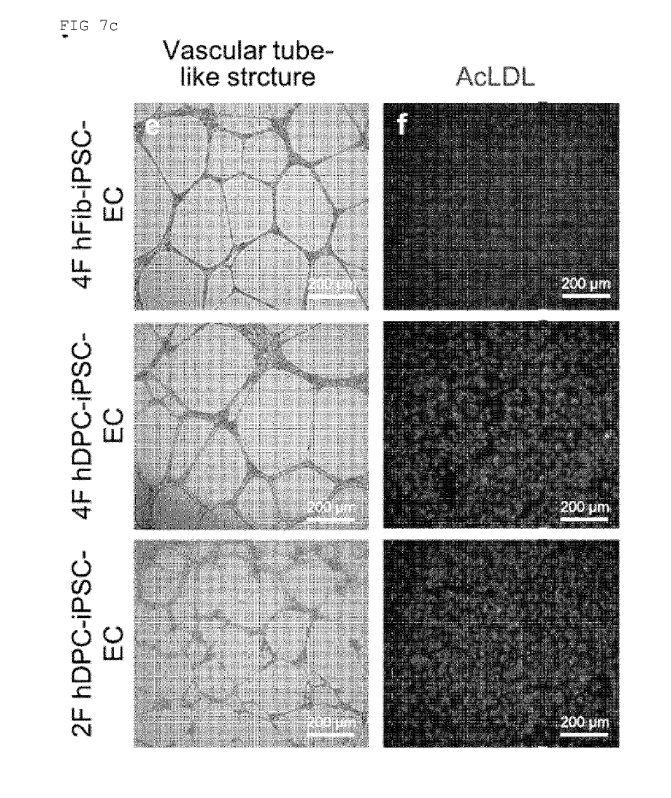

(2) As a result of the flow cytometry, after 20 days of EC differentiation, the cells from 2F hDPC-iPSC-CD34⁺ cells expressed typical endothelial markers, such as CD31 (99.1%) and CD105 (98.4%), at higher levels compared with those from 4F hFib-iPSC-CD34⁺ (CD31 (89.5%) and CD105 (87.1%)) and 4F hDPC-iPSC-CD34⁺ (CD31 (78.7%) and CD105 (76.2%)) (FIG. 7a). The EC-like properties of the differentiated cells were further characterized according to their typical cobblestone-like morphology and the increased expression of endothelial markers, such as vWF, VEcadherin, and CD31, as confirmed through immunohistochemistry (FIG. 7b).

Additionally, according to example 4-6, the functional features of ECs from hDPC-iPSCs (hDPC-iPSC-ECs) were also confirmed through the formation of vascular-like structures on Matrigel and the uptake of acetylated-LDL (FIG. 7c).

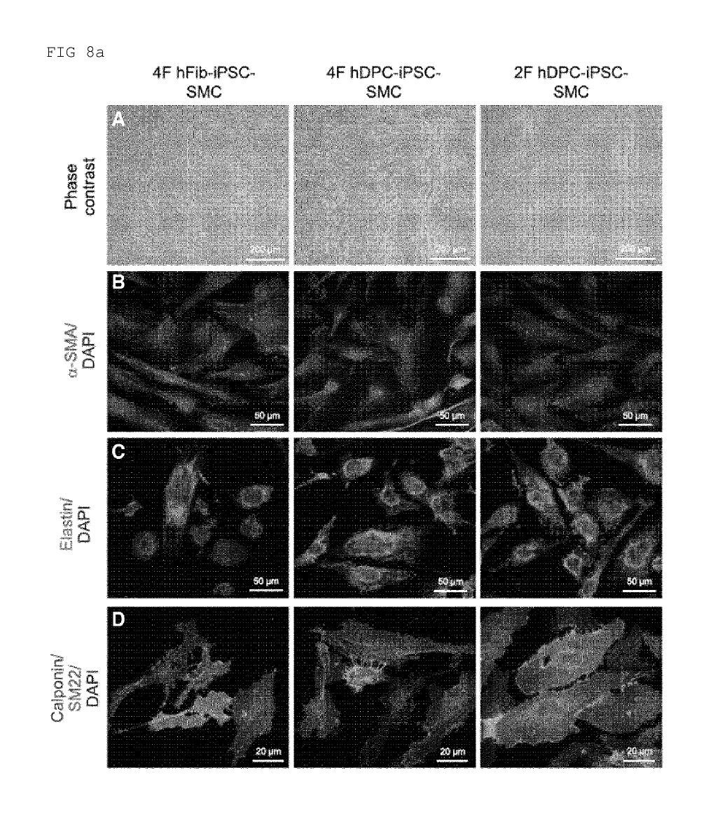

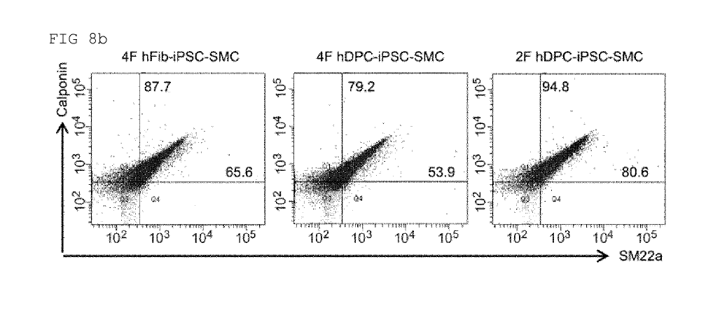

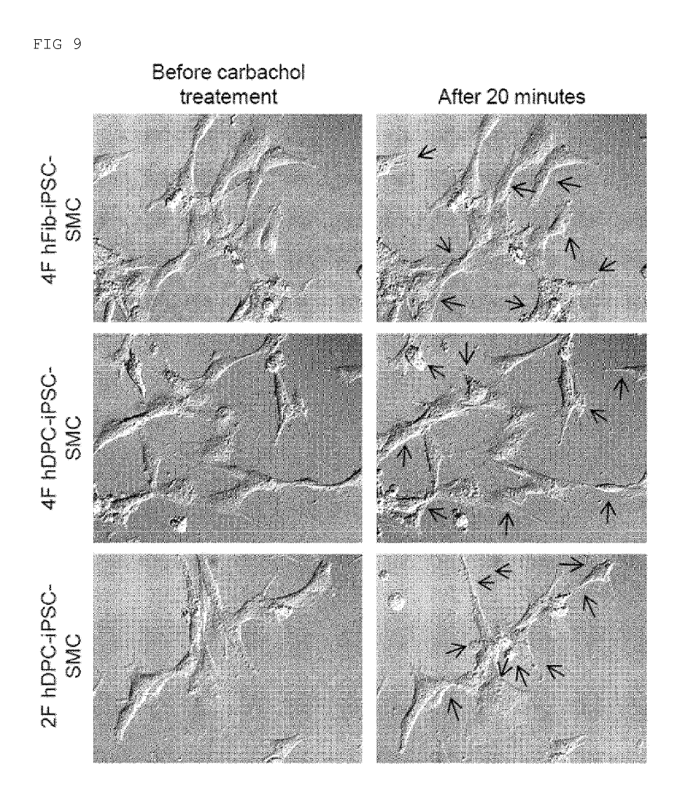

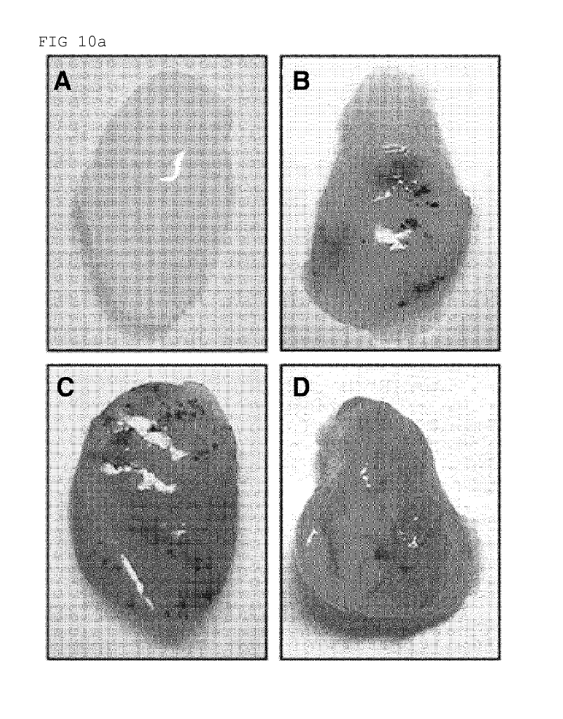

(3) After 20 days of SMC differentiation, the cells displayed SMC-like properties, such as a spindle-like morphology and the strong expression of smooth muscle-specific markers, including α-SMA, elastin, SM22, and calponin, as confirmed through immunohistochemistry (FIG. 8a).

Flow cytometry analysis of example 4-4 confirmed that calponin⁺ (94.8%) and SM22⁺ (80.6%) SMCs were notably higher in cell populations differentiated from 2F hDPC-iPSCs compared with those from 4F hFib-iPSCs (calponin (87.7%) and SM22a (65.6%)) and 4F hDPC-iPSCs (calponin (79.2%) and SM22a (53.9%)) (FIG. 8b).

In addition, hiPSC-derived SMCs (hiPSC-SMCs) were contracted within 30 min after treatment with carbachol, an agonist against acetylcholine receptors (FIG. 9), confirming the functionality of hiPSC-SMCs.

These results indicate that CD34⁺ cells derived from hDPC-iPSCs have great potential to produce patient-specific functional ECs and SMCs.

### Example 5

### In Vivo Matrigel Plug Assay

To evaluate the ability of hDPC-iPSC-CD34⁺ cells in angiogenesis in vivo, the Matrigel implant assay was employed in mice. Seven-week-old male nude mice (BalB/cAnNCriBgi-nu; Orient Bio, Korea) were subcutaneously injected with 0.6 ml of Matrigel containing 1×10⁶ hiPSC-derived CD34⁺ cells (hiPSC-CD34⁺ cells). The injected Matrigel rapidly formed a single, solid plug. After seven days, the Matrigel plugs were surgically excised from the mice without connective tissues. Quantification of blood vessel formation was measured using the Drabkin Reagent Kit 525 (Sigma). The hemoglobin concentration was estimated by comparing to a known amount of hemoglobin in parallel using an ELISA reader. In addition, the excised Matrigel was embedded into paraffin and sectioned for immunohistochemistry. The ECs in the Matrigel plugs were identified through immunostaining using mouse anti-CD31 and human anti-CD31 antibodies. Incorporated MACS-sorted CD34⁺ EPCs and mouse ECs were visualized using confocal microscopy. The total number of mouse CD31⁺ cells per field for each Matrigel sample was manually counted.

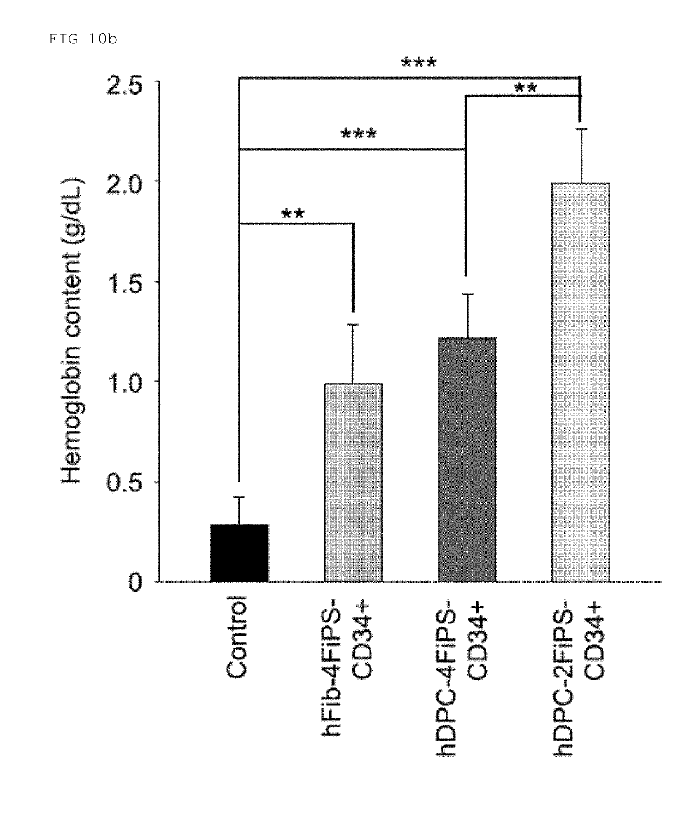

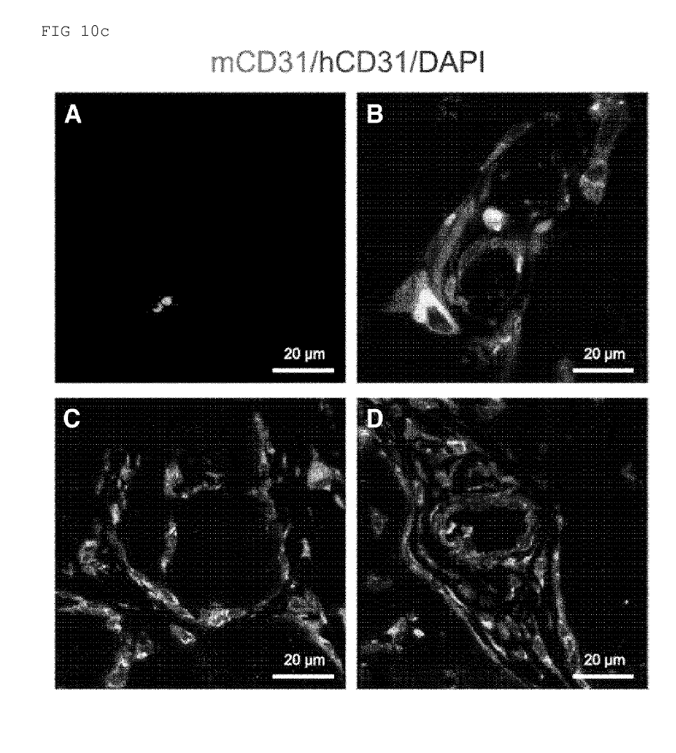

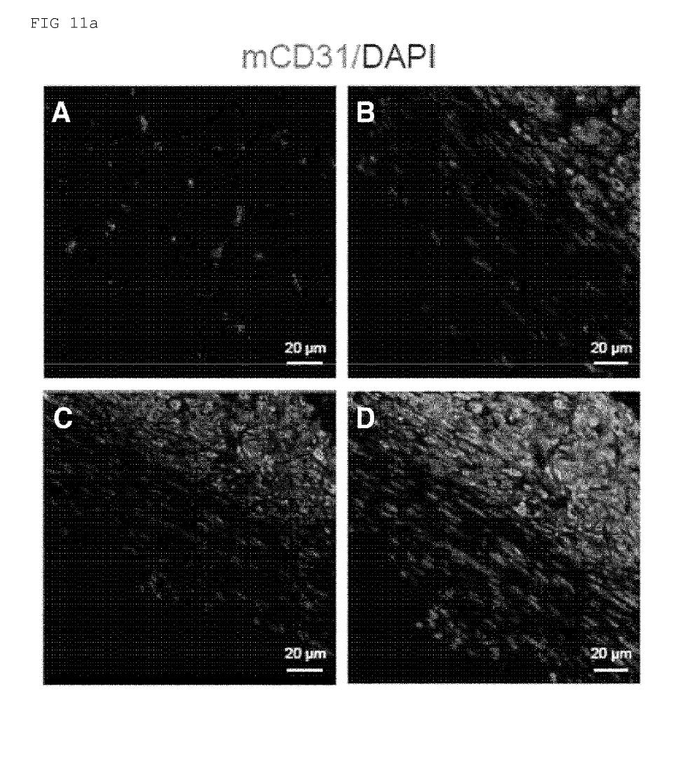

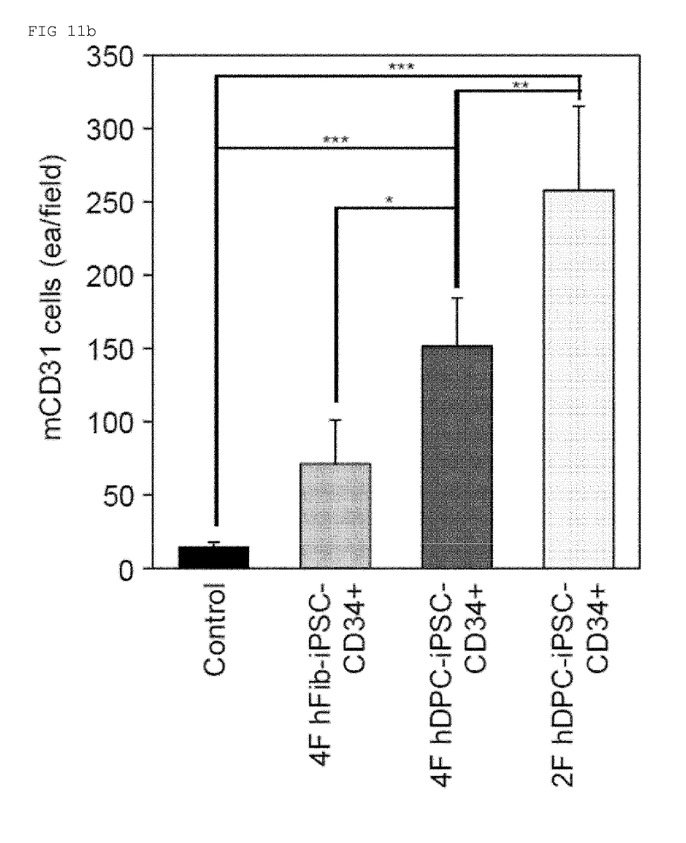

As shown in FIG. 10a, the implanted Matrigel turned red in the presence of CD34⁺ cells (FIGS. 10aB, 10aC, and 10aD), indicating an abundance of red blood cells in the newly formed vessels, whereas the plugs with Matrigel alone remained white (FIG. 10aA). The quantification of hemoglobin in the Matrigel plugs consistently indicated that hDPC-iPSC-CD34⁺ cells significantly increased angiogenesis (FIG. 10b). To further assess the neovasculogenesis of hDPC-iPSC-CD34⁺ cells, an immunohistochemical analysis of mouse CD31⁺ microvessels was performed (FIG. 10c and FIG. 11). The incorporation (arrow) between human and mouse vascular cells was observed in the hDPC-iPSC-CD34⁺ cell transplants, showing that hDPC-iPSC-CD34⁺ cells could differentiate into ECs and cooperate with native ECs in vivo (FIG. 10c). In other words, the EPCs can induce angiogenesis in vivo.

The immunohistochemical analysis confirmed a significantly higher population of mouse CD31⁺ cells in the Matrigel plugs containing hiPSC-CD34⁺ cells than in the untransplanted controls (FIGS. 11a and 11b). These results indicate that hDPC-iPSC-CD34⁺ cells, which have neoangiogenic and neovasculogenic potential, directly and indirectly contribute to neovessel formation. Moreover, the CD34⁺ cells derived from 2F hDPC-iPSCs exhibited higher angiogenic activity than those derived from 4F hDPC-iPSCs and 4F hFib-iPSCs.

### Example 6

### Hind-Limb Ischemia Model

Hind-limb ischemia was induced in BALB/cAnNCrljOri male mice (19-24 g) (Orient Bio) at 6-8 weeks of age through the ligation and excision of the right femoral artery and vein under ketamine-xylazine anesthesia. To determine the therapeutic effects, the mice were divided into four groups, and CD34⁺ cells from each hiPSCs were injected intramuscularly (l×10⁶ cells/20 μl medium) or DMEM (20 μl) as a control into each group. Time-series indocyanine green (ICG) perfusion imaging was performed immediately after surgery (day 0) and on post-operative days (POD) 3 and 7. For ICG imaging, mice under ketamine-xylazine anesthesia were injected with an intravenous bolus of ICG (0.1 ml of 400 μmol/L, Sigma) into the tail vein. ICG fluorescence images were obtained using customized optical systems for near-infrared (NIR) fluorescence imaging in a dark room for 5 min at 1-s intervals immediately after injection. After serial imaging, the perfusion rate of each pixel was calculated and translated into a pseudo color-coded perfusion rate map using Visual C⁺⁺ (version 6.0, Microsoft, USA). In addition, Visual C⁺⁺ was used to obtain perfusion maps and necrosis probabilities.

For the histological analysis, the mice were anesthetized through intramuscular injection (80 mg/kg ketamine and 12 mg/kg xylazine) and subsequently fixed through the vascular perfusion of 1% paraformaldehyde in PBS. The tissues were harvested and embedded in cryofreezing medium. The cryosections (12 μm-thickness) were incubated in PBST (0.3% Triton X-100 in PBS) containing 5% donkey serum (Jackson ImmunoResearch Laboratories) at RT for 1 h. After blocking, the samples were incubated at 4° C. overnight with mouse anti-human and anti-mouse CD31 antibodies (Abcam, 1:200). After several washes in PBST, the samples were incubated with fluorescent-conjugated secondary antibodies (Molecular probes, 1:500) for 1 h at RT. The nuclei were stained with DAPI at RT for 10 min. The signals were visualized using a FluoView confocal microscope (Olympus).

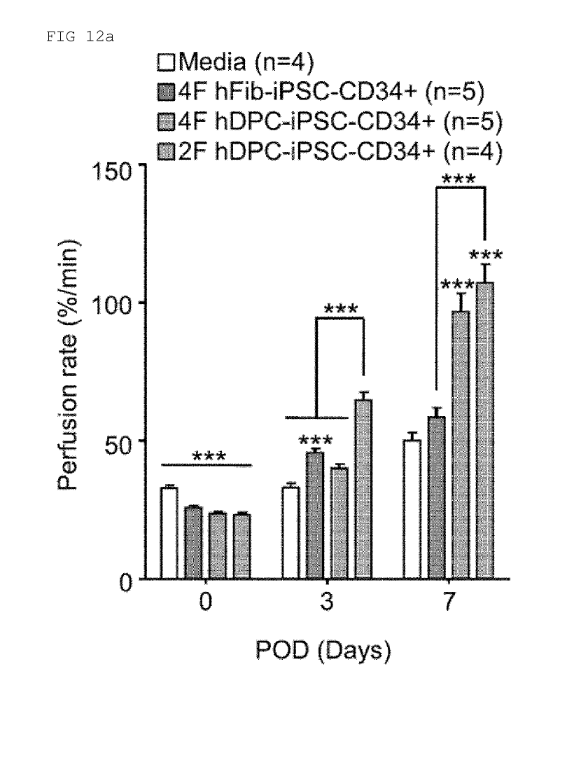

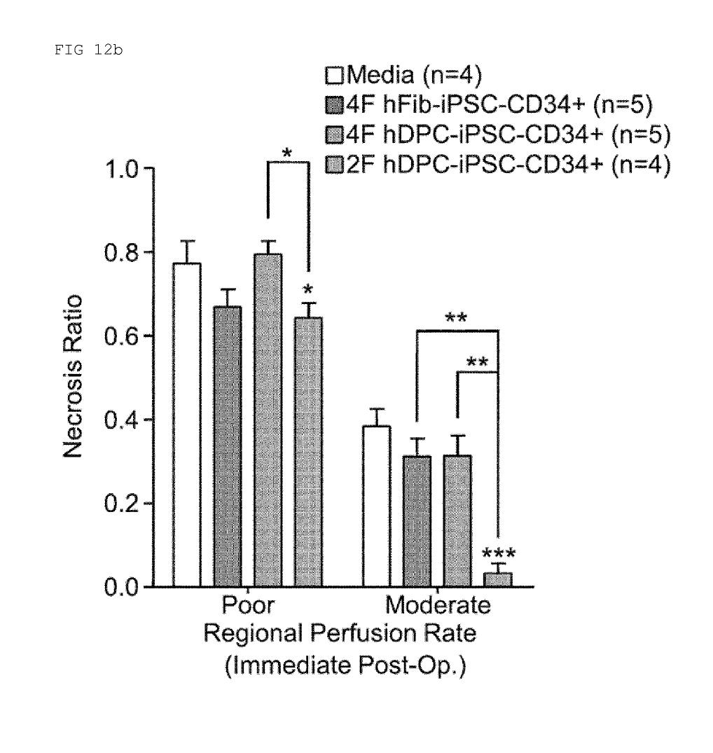

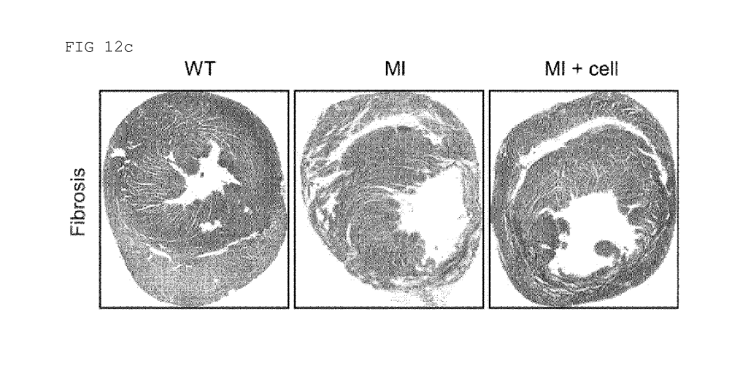

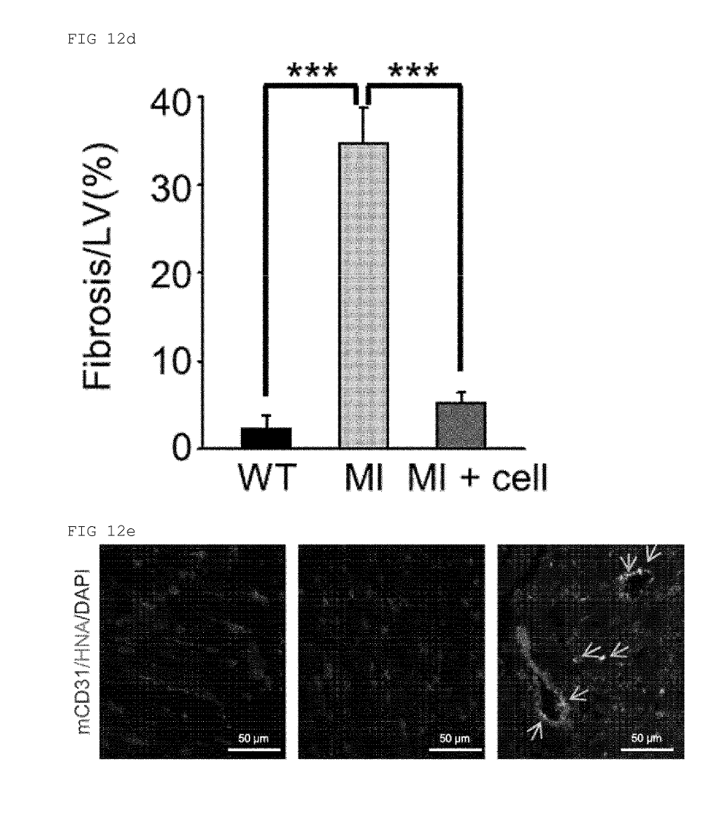

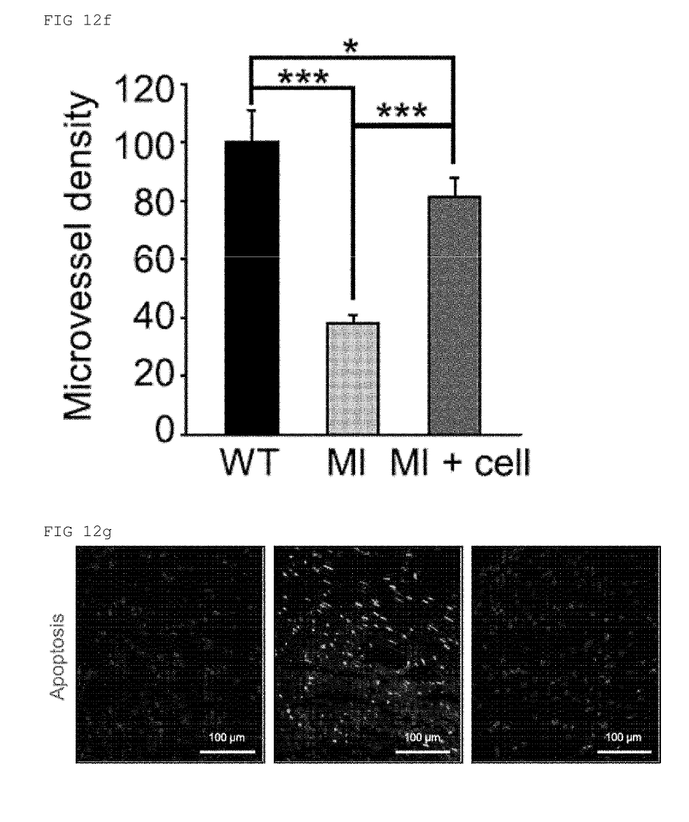

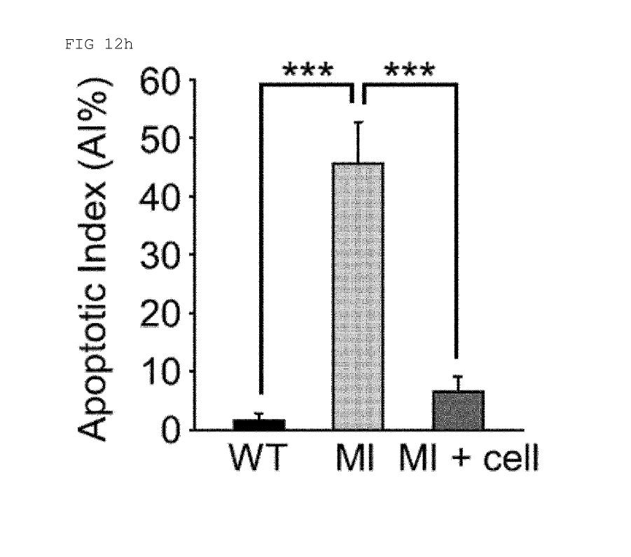

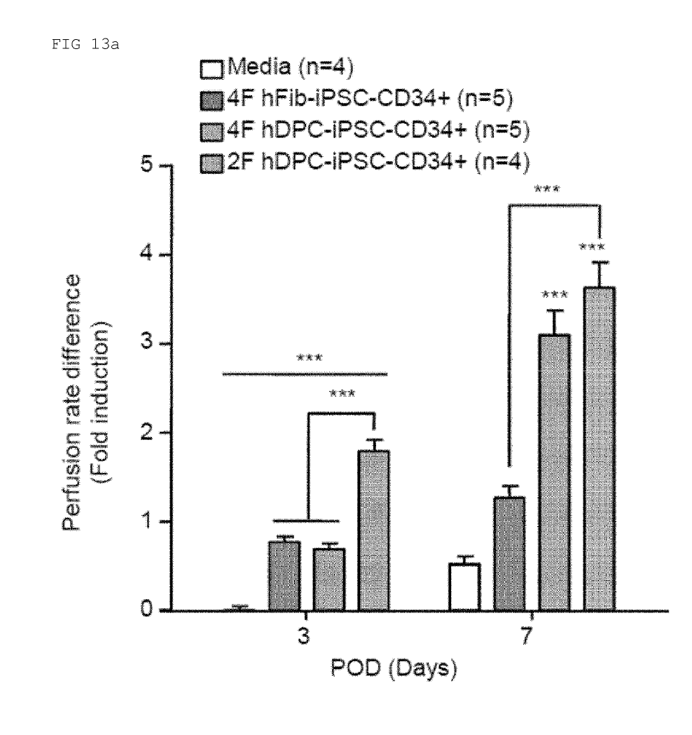

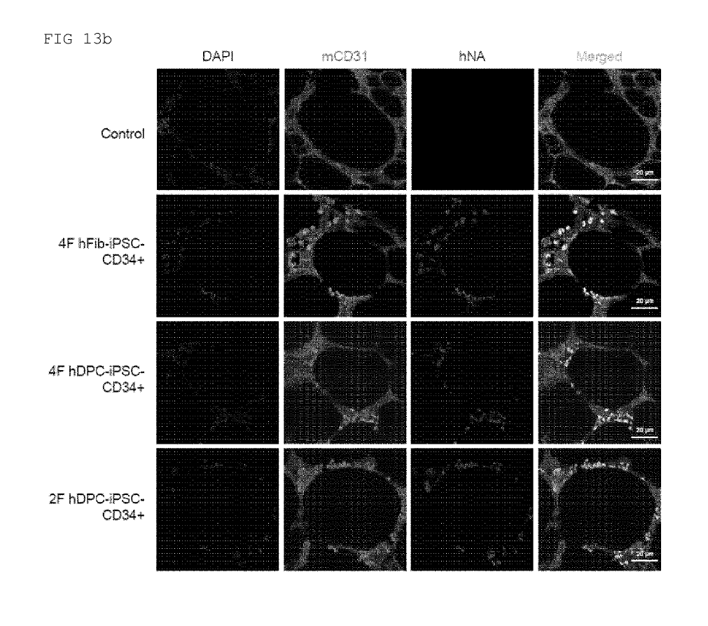

Intergroup comparisons of time-dependent perfusion rates clearly demonstrated the therapeutic effects of CD34⁺ cells from hiPSCs, particularly between POD 3 and 7 (FIG. 12a, and FIG. 13a). CD34⁺ cells from 2F hDPC-iPSCs were more effective in the restoration of perfusion in ischemic hind-limbs (FIG. 12a, and FIG. 13a) compared with those from 4F hDPC-iPSCs and 4F hFib-iPSCs. Also, CD34⁺ cells from 2F hDPC-iPSCs were the most effective in reducing limb necrosis (FIG. 12b).

To determine whether transplanted hiPSC-CD34⁺ cells recover the ischemic hind-limb, tissue samples were harvested from the operated region on POD 7. Coexistence of human nuclear antigen (hNA) and mouse CD31⁺ cells was observed in ischemic hind-limb regions injected with hiPSC-CD34⁺ cells (FIG. 13b), suggesting that hiPSC-CD34⁺ cells develop into ECs in vivo, thereby contributing to neovasculogenesis in the ischemic region.

### Example 7

### Myocardial Infarction (MI) Model

The therapeutic effect of 2F hDPC-iPSC-CD34⁺ cells in a mouse model of MI was assessed. MI was induced in 8-week-old BALB/cAnNCrljOri male mice. The mice were anesthetized using ketamine and xylazine, and the surgical occlusion of the left anterior descending coronary artery was performed using a 6-0 silk suture (Johnson & Johnson, New Brunswick, N.J.). After occlusion, 1×10⁶ hiPSC-CD34⁺ cells in 20 μl media were injected into three sites around the occluded region. During the operation, the mice were ventilated with 95% O2 and 5% CO2 using a Harvard ventilator. Six animals per group were used for morphological analysis at 1 week post-operation.

For histological analysis, the heart was surgically removed, fixed with 10% PBS buffered formalin for 24 h, and embedded in paraffin. The sections (5 μm-thick) were mounted on glass slides, deparaffinized and rehydrated. The tissue sections were stained with Masson's trichrome for the analysis of fibrosis. For immunohistochemical analysis, the sections were deparaffinized, rehydrated, and rinsed with PBS. For antigen retrieval, the samples were microwaved for 10 min in 10 mM sodium citrate (pH 6.0). The sections were incubated in 3% H₂O₂ to quench endogenous peroxidase activity. The sections were blocked in 3% normal bovine serum and incubated with a primary antibody against CD31/PECAM-1 (Abcam, 1:200) and human nuclear antigen (hNA) (Millipore, 1:200). After several washes in PBST, the samples were incubated with Alexa fluor 488 or 568 fluorescent-conjugated secondary antibodies for 1 h at RT. DAPI counterstaining was performed to allow for visualization of nuclei.

The TUNEL assay was performed according to the manufacturer's instructions (Chemicon International, Temecula, Calif., USA). A normal heart section treated with DNase I (10 U/ml, min at RT) was used for the positive control. After pretreatment with 3.0% H₂O₂, the sections were treated with a TdT enzyme at 37° C. for 1 h and incubated in a digoxigenin-conjugated nucleotide substrate at 37° C. for 30 min. DNA fragmentation in nuclei were visualized after treatment of 3,3-diaminobenzidine (DAB; Vector Laboratories) for 5 min. Subsequently, the sections were counterstained with methyl green to observe apoptosis in the tissue sections using light microscopy.

One week after the induction of MI and injection of CD34⁺ cells, the size of the left ventricular (LV) infarct was evaluated in both the transplanted and untransplanted groups. The injection of CD34⁺ cells significantly reduced the infarct size and the degree of fibrosis in the infarct zone compared with the controls (FIGS. 12c and 12d).

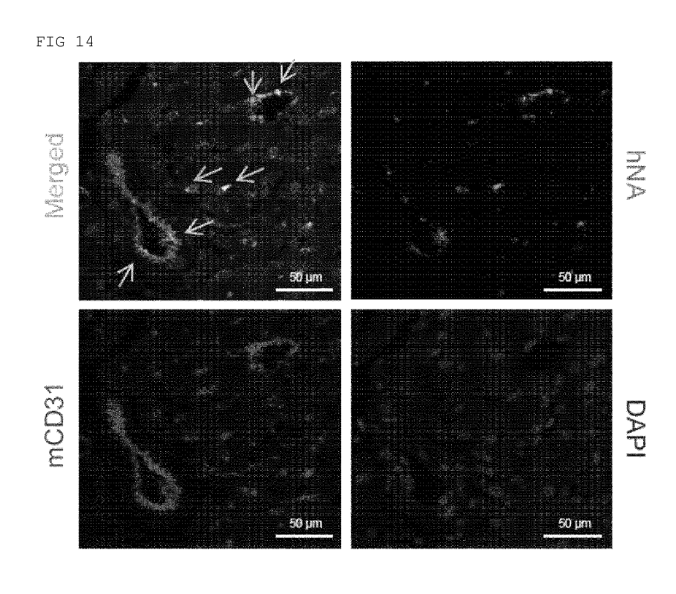

2F hDPC-iPSC-CD34⁺ cells effectively restored the density of CD31⁺ microvessels in the infracted myocardial tissues (FIGS. 12e and 12f). In addition, mouse CD31 and hNA double staining showed incorporation into newly formed vessels (FIG. 12e and FIG. 14). The incidence of TUNEL⁺ myocardial cells was significantly reduced in the CD34⁺ cell group compared with the controls (FIGS. 12g and 12h). These observations indicate that hDPC-iPSC-CD34⁺ cells are involved, either directly or indirectly, in neovasculogenesis, ameliorating MI-induced cardiac dysfunction in mice.

### Example 8

### Primers and Antibodies

The primer sequences used for PCR amplification are listed in Table 1. The antibodies used and dilution factors are described in Table 2.

TABLE 1

| | | | |
|---|---|---|---|
| | | | Accession |
| Gene | Sequences | Size | No. |

Genomic DNA integration

| | | | |
|---|---|---|---|
| Trans | F: GAGAAGGATGTGGTCCGAGTGTG | 475 | NM_002701.4 |
| OCT4 | (Sequence ID NO 1) | | |
| | R: CCCTTTTTCTGGAGACTAAATAAA | | |
| | (Sequence ID NO 2) | | |
| Trans | F: GGCACCCCTGGCATGGCTCTTGGCTC | | |
| SOX2 | (Sequence ID NO 3) | 720 | NM_003106.2 |
| | R: TTATCGTCGACCACTGTGCTGCTG | | |
| | (Sequence ID NO 4) | | |
| Trans | F: ACGATCGTGGCCCCGGAAAAGGACC | 484 | NM_004235.4 |
| KLF4 | (Sequence ID NO 5) | | |
| | R: TTATCGTCGACCACTGTGCTGCTG | | |
| | (Sequence ID NO 6) | | |
| Trans | F: CAACAACCGAAAATGCACCAGCCCCAG | | |
| c-Myc | (Sequence ID NO 7) | 301 | NM_002467.3 |
| | R: TTATCGTCGACCACTGTGCTGCTG | | |
| | (Sequence ID NO 8) | | |

DNA methylation

| | | | |
|---|---|---|---|
| biOCT4-3 | F: ATTTGTTTTTTGGGTAGTTAAAGGT | 220 | NM_002701.4 |
| | (Sequence ID NO 9) | | |
| | R: CCAACTATCTTCATCTTAATAACATCC | | |
| | (Sequence ID NO 10) | | |
| biOCT4-4 | F: GGATGTTATTAAGATGAAGATAGTTGG | 354 | NM_002701.4 |
| | (Sequence ID NO 11) | | |
| | R: CCTAAACTCCCCTTCAAAATCTATT | | |
| | (Sequence ID NO 12) | | |
| biNanog | F: TGGTTAGGTTGGTTTTAAATTTTTG | 336 | NM_024865 |
| | (Sequence ID NO 13) | | |
| | R: AACCCACCCTTATAAATTCTCAATTA | | |
| | (Sequence ID NO 14) | | |

Mesodermal lineage markers

| | | | |
|---|---|---|---|
| IGF2 | F: CAGACCCCCAAATTATCGTG | 208 | NM_000612 |
| | (Sequence ID NO 15) | | |
| | R: GCCAAGAAGGTGAGAAGCAC | | |
| | (Sequence ID NO 16) | | |
| MSX1 | F: CGAGAGGACCCCGTGGATGCAGAG | 307 | NM_002448.3 |
| | (Sequence ID NO 17) | | |
| | R: GGCGGCCATCTTCAGCTTCTCCAG | | |
| | (Sequence ID NO 18) | | |
| Brachyury | F: GCCCTCTCCCTCCCCTCCACGCACAG | 274 | NM_003181.3 |
| | (Sequence ID NO 19) | | |
| | R: CGGCGCCGTTGCTCACAGACCACAGG | | |
| | (Sequence ID NO 20) | | |
| Runx2 | F: CGGCAAAATGAGCGACGTG | 268 | NM_004348 |
| | (Sequence ID NO 21) | | |
| | R: CACCGAGCACAGGAAGTTG | | |
| | (Sequence ID NO 22) | | |

Endothelial cell-specific markers

| | | | |
|---|---|---|---|
| CD31 | F: ATCATTTCTAGCGCATGGCCTGGT | 159 | NM_000442.4 |
| | (Sequence ID NO 23) | | |
| | R: ATTTGTGGAGGGCGAGGTCATAGA | | |
| | (Sequence ID NO 24) | | |
| CD34 | F: AAATCCTCTTCCTCTGAGGCTGGA | 216 | NM_001773.2 |
| | (Sequence ID NO 25) | | |
| | R: AAGAGGCAGCTGGTGATAAGGGTT | | |
| | (Sequence ID NO 26) | | |
| VE-cad | F: TGGAGAAGTGGCATCAGTCAACAG | 118 | NM_001795.3 |
| | (Sequence ID NO 27) | | |
| | R: TCTACAATCCCTTGCAGTGTGAG | | |
| | (Sequence ID NO 28) | | |
| KDR | F: ACACCTAAAAACCCAGCAC | 145 | NM_002253.2 |
| | (Sequence ID NO 29) | | |
| | R: TCTGATTCCTTCTTCTTCAT | | |
| | (Sequence ID NO 30) | | |

Housekeeping gene

| | | | |
|---|---|---|---|
| GAPDH | F: GAAGGTGAAGGTCGGAGTC | 226 | NM_002046 |
| | (Sequence ID NO 31) | | |
| | R: GAAGATGGTGATGGGATTTC | | |
| | (Sequence ID NO 32) | | |

TABLE 2

| | | | |
|---|---|---|---|
| | Catalog | | |
| Antibodies | No. | Company | Dilution |

Pluripotency marker

| | | | |
|---|---|---|---|
| anti-Oct4 | sc-9081 | Santa Cruz | 1:100 |
| | | Biotechnology | |
| anti-Nanog | AF1997 | R&D | 1:40 |
| anti-SSEA3 | MAB1434 | R&D | 1:30 |
| anti-SSEA4 | MAB1435 | R&D | 1:30 |
| anti-Tra1-60 | MAB4360 | Chemicon | 1:100 |
| anti-Tra1-81 | MAB4381 | Chemicon | 1:100 |

Differentiation marker

| | | | |
|---|---|---|---|
| anti-Tuj1 | PRB-435P | Covance | 1:500 |
| anti-Nestin | MAB5326 | Chemicon | 1:100 |
| anti-α-SMA | A5228 | Sigma | 1:200 |
| anti-Desmin | AB907 | Chemicon | 1:50 |
| anti-Sox17 | MAB1924 | R&D | 1:50 |
| anti-Foxa2 | 07-633 | Millipore | 1:100 |

Endothelial cell-specific marker

| | | | |
|---|---|---|---|
| human anti-CD31 | AB76533 | Abcam | 1:100 |
| mouse anti-CD31 | AB9498 | Abcam | 1:100 |
| anti-vWF | B11713 | Abcam | 1:500 |
| anti-VE-cad | AB7047 | Abcam | 1:500 |

Smooth muscle cell-specific marker

| | | | |
|---|---|---|---|
| anti-Calponin | C2687 | Sigma | 1:1000 |
| anti-SM22 | AB10135 | Abcam | 1:1000 |
| anti-Elastin | AB21610 | Abcam | 1:1000 |

Secondary antibodies

| | | | |
|---|---|---|---|
| anti-rabbit IgG- | A21442 | Invitrogen | 1:200 |
| Alexa-594 | | | |
| anti-goat IgG- | A21467 | Invitrogen | 1:200 |
| Alexa-488 | | | |
| anti-mouse IgM- | A21044 | Invitrogen | 1:200 |
| Alexa-594 | | | |
| anti-rat IgG-FITC | 553887 | BD pharmingen | 1:200 |
| anti-mouse IgG- | A11001 | Invitrogen | 1:200 |
| Alexa-488 | | | |

### Example 9

### Statistical Analysis

The data is expressed as the means±SEM (standard error of the mean). Differences between the groups were analyzed using Student's t-test. A p-value≦0.05 was considered significant. The data is representative of at least three independent experiments.

From the above-mentioned embodiments, it may be understood by those skilled in the art to which the present invention pertains that the present invention may be implemented in other specific forms without changing the spirit or essential features thereof. Therefore, it should be understood that the above-mentioned embodiments are not restrictive but are exemplary in all aspects. The scope of the present invention should be defined by the meaning and the scope of the appended claims to be described below as well as all alterations or modified forms from the equivalent concepts rather than the above detailed explanations.

What is claimed is:

1. A method for producing induced pluripotent stem cells from human dental pulp cells, comprising using only Oct4 and Sox2 as a reprogramming factor.

2. The method of claim 1, comprising:
(a) delivering genes of Oct4 and Sox2 into human dental pulp cells;
(b) culturing the cells of (a); and
(c) purifying induced pluripotent stem cells from the cultured cells.

3. The method of claim 2, wherein nicotinamide is added to said culturing in step (b).

4. The method of claim 2, wherein step (a), step (b), and step (c) are simultaneously or sequentially performed, or step (b) and step (c) are repeatedly performed.

5. A method for producing endothelial progenitor cells, comprising differentiating induced pluripotent stem cells into endothelial progenitor cells, wherein the induced pluripotent stem cells are produced by the method of claim 1.

6. The method of claim 5, wherein the differentiating comprises culturing the induced pluripotent stem cells in a medium comprising BMP4 (bone morphogenetic protein 4), Activin A, VEGF-A (vascular endothelial growth factor-A) and bFGF (basic fibroblast growth factor).

7. A method for producing endothelial cells, comprising differentiating endothelial progenitor cells into endothelial cells, wherein the endothelial progenitor cells are produced by the method of claim 5.

8. The method of claim 7, wherein the differentiating comprises culturing the endothelial progenitor cells in a medium comprising VEGF-A and bFGF.

9. A method for producing smooth muscle cells, comprising differentiating endothelial progenitor cells into smooth muscle cells, wherein the endothelial progenitor cells are produced by the method of claim 5.

10. The method of claim 9, wherein the differentiating comprises culturing the endothelial progenitor cells in a medium comprising bFGF and PDGF (platelet-derived growth factor-BB).

11. The induced pluripotent stem cells having an enhanced rate of differentiation into endothelial progenitor cell, wherein the induced pluripotent stem cell is produced by the method of claim 1.

12. The induced pluripotent stem cells having an enhanced rate of differentiation into endothelial progenitor cells, wherein the induced pluripotent stem cells are produced by the method comprising:
(a) delivering genes of Oct4 and Sox2 into human dental pulp cells;
(b) culturing the cells and adding nicotinamide; and
(c) purifying induced pluripotent stem cells from the cultured cells.

13. The endothelial progenitor cells having an enhanced rate of differentiation into smooth muscle cells or endothelial cells, wherein the endothelial progenitor cells are produced by the method of claim 6.

14. The endothelial cells produced by the method of claim 8.

15. The smooth muscle cells produced by the method of claim 10.

16. A method for producing a composition for treating ischemic disease, comprising:
(a) preparing induced pluripotent stem cells produced by the method of claim 1;
(b) preparing endothelial progenitor cells differentiated from the induced pluripotent stem cells;
(c) preparing endothelial cells differentiated from the endothelial progenitor cells; or
(d) preparing smooth muscle cells differentiated from the endothelial progenitor cells;
wherein the composition comprises induced pluripotent stem cells, endothelial progenitor cells, endothelial cells, smooth muscle cells, or a combination thereof, which are prepared in any one of steps (a) through (d).

17. A method for producing a composition for treating heart disease, comprising:
(a) preparing induced pluripotent stem cells produced by the method of claim 1;
(b) preparing endothelial progenitor cells differentiated from the induced pluripotent stem cells;
(c) preparing endothelial cells differentiated from the endothelial progenitor cells; or
(d) preparing smooth muscle cells differentiated from the endothelial progenitor cells;
wherein the composition comprises induced pluripotent stem cells, endothelial progenitor cells, endothelial cells, smooth muscle cells, or a combination thereof, which are prepared in any one of steps (a) through (d).

18. A method of treatment for ischemic disease comprising administering the composition produced by the method of claim 16 to an individual requiring the composition.

19. A method of treatment for heart disease comprising administering the composition produced by the method of claim 17 to an individual requiring the composition.
